(19) United States
(12) Patent Application Publication
(10) Pub. No.: US 2008/0132450 A1
(43) Pub. Date: Jun. 5, 2008

(54) Pharmaceutical composition for suppression of apoptosis and method for delivering the same

(75) Inventors: Sang-Kyou Lee, Seoul (KR); Seung-Kyou Lee, Kyeunggi-Do (KR); Yang-Soo Jang, Seoul (KR); Ki-Chul Hwang, Seoul (KR)

Correspondence Address: STERNE, KESSLER, GOLDSTEIN & FOX P.L.L.C., 1100 NEW YORK AVENUE, N.W., WASHINGTON, DC, 20005, US

(73) Assignee: ForHumanTech. Co., Ltd.

(21) Appl. No.: 11/892,947

(22) Filed: Aug. 28, 2007

Related U.S. Application Data
(60) Provisional application No. 60/840,697, filed on Aug. 29, 2006.

(51) Int. Cl. A61K 38/17; C07K 14/435; C12N 5/02; A01N 1/02; A61P 25/00; A61P 9/00; A61K 35/12

(52) U.S. Cl. 514/12; 530/350; 435/375; 435/1.1; 424/93.7

(57) ABSTRACT

The present invention relates to a pharmaceutical composition for treating heart diseases, neurodegenerative diseases, and diseases and conditions caused by apoptosis, which contains a conjugate of a heat shock protein (Hsp) and a protein transduction domain (PTD). According to the present invention, PTD-Hsp70 effectively suppresses apoptosis under low-oxygen conditions.

### CROSS-REFERENCE TO RELATED APPLICATION

This application claims the benefit of U.S. Provisional Patent Application No. 60/840,697, filed Aug. 29, 2006.

### BACKGROUND OF THE INVENTION

1. Field of the Invention

The present invention relates to a novel pharmaceutical composition for treating heart diseases, neurodegenerative diseases, and diseases and conditions caused by apoptosis, which contains a conjugate of a molecule of interest, such as a heat-shock-protein (Hsp), and a protein transduction domain (PTD), as well as a method for delivering the same.

2. Background Art

Apoptosis, also called “programmed cell death,” is a mechanism in which cells destroy themselves, when the cells undergo various signal stimulations, i.e., when the cells are no longer needed or represent a threat to the integrity of the organism. Apoptosis is an active and well-regulated process, which is required not only for maintaining the life of adult individuals but also during embryogenesis, morphogenesis and metamorphosis, and is associated with cell death caused by hormones and various chemicals. If apoptosis occurs at an unsuitable time, or if essential apoptosis is inhibited, various diseases, such as cancer and autoimmune diseases, can occur.

Apoptosis results in various phenomena, including the condensation of nucleic acid and the breakdown of DNA to a constant size, as well as changes in intracellular organelles, endoplasmic reticulum, cellular membrane and the like. Also, it progresses such that dead cells can be removed by phagocytosis without adversely affecting the surrounding cells.

Apoptosis of isolated organs for transplantation can also occur. The prevention of apoptosis in isolated organs increases the success rate of organ transplantation. A preservation solution for preserving isolated organs until transplantation is an important factor for increasing the success rate of organ transplantation. Organ preservation solutions which are currently widely used include Viaspan™, the University of Wisconsin solution, HTK™ (histidine-tryptophan ketoglutarate solution), SGF (silica gel filtered plasma) and the like.

Reperfusion, although generally considered beneficial, causes tissue injury by several mechanisms. Clinically, in open heart surgery, heart transplantation, and reversal of heart disease, protection of the myocardium against injury by ischemia-reperfusion is an issue of utmost clinical interest. Exacerbation of hypoxic injury after restoration of oxygenation (reoxygenation) by reperfusion is an important mechanism of cellular injury in other types of organ transplantation and in hepatic, intestinal, cerebral, renal, and other ischemic syndromes.

The composition of the organic preservation solution is an important factor, and recently, in addition to factors for preventing the drying of organs and maintaining the osmotic pressure of organs, methods of adding various compounds for inhibiting the apoptosis of organs have been suggested.

Chaperones are a functionally related group of proteins that assist protein folding in bacteria, plant and animal cells under physiological and stress conditions. (Giffard, R. G., et al., J. Exp. Biol. 207:3213-3220 (2004)). Chaperones also facilitate translocation of protein complexes, help present substrates for degradation, and suppress protein aggregation. An important subgroup of highly conserved chaperones is the ATP-dependent heat-shock proteins (Hsps).

Under normal conditions, Hsps function as intracellular molecular chaperones of newly synthesized polypeptide chains, preventing their aggregation during folding and subunit assembly and during the translocation of proteins across subcellular membranes to their appropriate cellular compartments. Some Hsps are involved in the clearance of proteins that are improperly folded and proteins that are unfolded as a result of their decreased stability under conditions of cellular stress (for example, oxidation and high temperatures). In addition to stress-induced members, most Hsp families also contain members that are constitutively expressed.

Heat-shock protein 70 (Hsp70) is a highly conserved protein chaperone involved in a number of intracellular mechanisms. Hsp70 is induced by intracellular stress and suppresses stress-induced apoptosis. Hsp70 also has immunoregulatory potential and is known to stimulate the production of anti-inflammatory cytokines. (see Van Eden, W., et al., Nat. Rev. Immunol. 5:318-330 (2005)). In addition, it prevents inflammatory shock caused by tumor necrosis factor (TNF) and induces antigen presentation.

Members of the Hsp family, including Hsp70, are also known to regulate T-cells in chronic inflammatory diseases to prevent or interrupt apoptosis caused by inflammation (see Van Eden, W., et al., Nat. Rev. Immunol. 5:318-330 (2005)). For example, it was shown that an Hsp70-derived peptide induced protection against experimentally induced arthritis (Tanaka, S., et al., J. Immunol. 163:5560-5565 (1999)).

Neurodegenerative diseases such as Alzheimer's disease and Huntington's disease (polyglutamine disease) are typical diseases likely caused by the abnormal accumulation of misfolded and aggregated proteins, and these diseases are thought to be inhibited by the action of Hsp70 as a chaperone. Apoptosis is one of the ways neurons die after ischemia. It has been shown that overexpression of Hsp70 in hippocampal CA1 neurons reduces evidence of protein aggregation under conditions where neuronal survival is increased (Giffard, R. G., et al., J. Exp. Biol. 207:3213-3220 (2004)).

Ischemic and hypoxic apoptosis may also occur due to defective clearance of proteins that are improperly folded or unfolded as a result of their decreased stability under conditions of abnormal oxidation. It was reported that Hsp70 acts together with co-chaperone Hsp40 to suppress the ischemic or hypoxic apoptosis of cerebral astrocytes upon the lack of glucose or oxygen-glucose (see Giffard, R. G., et al., J. Exp. Biol. 207:3213-3220 (2004)). These in vitro injury models mimic some of the aspects of injury involved in ischemic damage during stroke.

Moreover, the function of Hsp70 in diabetes is also known, and radical-induced injury to pancreatic beta cells is suppressed by overexpression of Hsp70 (see Burkart, V., et al., JBC 275:19521-19528 (2000); and Margulis et al., Diabetes 40:1418-1422 (1994)).

Methods of effectively delivering macromolecules such as Hsp70 into cells in vitro or in vivo are desired. Generally, living cells are impermeable to macromolecules, such as proteins and nucleic acids. Only some substances having small size can pass through the membrane of living cells at a low rate and enter the intracellular cytoplasm, organelle or nucleus. Most macromolecules cannot enter cells, imposing limitations on treatment, prevention and diagnosis using such macromolecules. Since substances prepared for the purposes of treatment, prevention and diagnosis should be delivered into cells in an effective amount, various methods for delivering these substances to cells have been developed.

Methods for delivering macromolecules into cells in vitro include electroporation, membrane fusion with liposomes, high velocity bombardment with DNA-coated microprojectiles, incubation with calcium-phosphate-DNA precipitate, DEAE-dextran mediated transfection, infection with modified viral nucleic acids, and direct micro-injection into single cells. Recently, there have been attempts to deliver macromolecules into cells in vivo and in vitro using nanoparticles, but these methods are still in an early stage in terms of technical level and clinical effect. Also, these methods can typically deliver macromolecules into only some of the cells, and the time and efficiency of delivering the macromolecules into cells do not yet reach a stage that can be clinically applied. Also, these methods can have undesirable effects on a large number of cells other than the target cells. Accordingly, there is a need for a general method for effectively delivering physiologically active macromolecules into cells both in vivo and in vitro without damaging the cells.

As a result protein transduction domains (PTDs) were studied. Among studies of PTDs, the most frequently studied is a Tat protein which is a transcription factor of human immunodeficiency virus-1 (HIV-1) (see Schwarze S. R., et al., Science 285(5433):1569-1572 (1999)). This protein was found to more effectively pass through the cell membrane when it consisted of amino acid residues 47-57 (YGRKKRRQRRR) with a concentrated distribution of positively charged amino acids, compared to when it is a complete form consisting of 86 amino acids (see Fawell, S., et al., Proc. Natl. Acad. Sci. USA 91:664-668 (1994)). Other amino acid sequences found to serve as the PTD include amino acid residues 267-300 of the HSV-1 (herpes simplex virus type 1) VP22 protein (see Elliott, G., et al., Cell 88:223-233 (1997)), and amino acid residues 339-355 of the Drosophila ANTP (Antennapedia) protein (see Schwarze, S. R., et al., Trends Pharmacol Sci. 21:45-48 (2000)).

The technology for delivering substances into cells using PTDs allows the production of medical proteins having a natural structure and function by delivering recombinant medical and pharmacological proteins produced in bacteria into the desired animal cells.

### BRIEF SUMMARY OF THE INVENTION

One object of the invention is to effectively suppress apoptosis and the development of diseases caused by apoptosis, by delivering a heat shock protein (Hsp) polypeptide in vivo.

To achieve the above object, the present invention provides a conjugate of a PTD and a heat-shock polypeptide (PTD-Hsp). The PTD-Hsp conjugate according to the present invention easily passes through membranes due to the intracellular penetration and delivery effects of PTD, for delivery to cells.

One embodiment of the present invention is a method of reducing or inhibiting apoptosis of a cell population whereby a cell population is contacted with an effective amount of PTD-Hsp.

A further embodiment is a method of treating, preventing or suppressing a pathological condition characterized by an elevated level of apoptosis, by administering to an individual in need of such treatment an amount of PTD-Hsp effective for treating the pathological condition.

An additional embodiment of the invention is a method of regenerating damaged cells, comprising storing the cells in an effective amount of PTD-Hsp.

Another embodiment of the invention is a method for expanding or increasing survival of a cell population by contacting the cells with an inhibiting and/or suppressing amount of PTD-Hsp.

A further embodiment of the invention is a method for prolonging cell, tissue or organ viability comprising contacting a cell population, tissue or organ with an inhibiting or suppressing amount of PTD-Hsp.

The invention also includes a method of increasing bioproduction in vitro whereby host cells that produce a product of interest are contacted with PTD-Hsp.

For all of the above embodiments, fusions of PTD with one or more fragments, derivatives or analogues of Hsp are also contemplated.

This invention also enables administration of the PTD-Hsp conjugate via local administration routes, thereby minimizing or avoiding systemic side effects.

### BRIEF DESCRIPTION OF THE DRAWINGS/FIGURES

FIG. 1A shows the expressed HspA1A and PTD-HspA1A proteins. Isolated and purified HspA1A and PTD-HspA1A fusion protein were subjected to SDS-PAGE, and analyzed by Coomassie blue staining. The molecular weight (mw) of HspA1A is about 70 kDa and the mw of PTD-HspA1A is about 72 kDa.

FIG. 1B shows 1 PTD-HspA1A expression in Jurkat T cells. One μl of proteins was added to a medium with Jurkat T cells and cultured for 1 hour. Only the PTD-conjugated protein was introduced into the cells.

FIG. 1C shows PTD-HspA1A suppression of apoptosis in a concentration-dependent manner. Cells were treated with 0.5 μM staurosporin to induce apoptosis. Various concentrations of the PTD-HspA1A were added and the cells analyzed for the degree of apoptosis. Cell survival increased with increasing amounts of PTD-HspA1A. Con represents Jurkat T cell only, and STS represents staurosporin.

FIGS. 2A-E represent experiments whereby the PDT-HspA1A was introduced into mesenchymal stem cells (MSC) under low-oxygen conditions and examined for its apoptosis-suppressing effect. Various concentrations of the purified PTD-HspA1A were introduced into mesenchymal stem cells (MSC) (FIG. 2A).

FIG. 2B shows that the apoptosis of MSC under low-oxygen conditions (hypoxia) was suppressed in the presence of HspA1A as shown by an increased WST-1 signal.

FIG. 2C shows that the relative caspase-3 activity in MSC was suppressed in the presence of HspA1A under low-oxygen conditions.

FIG. 2D shows that ATP levels increased in MSC in the presence of HspA1A under low-oxygen conditions.

FIG. 2E shows that the introduction of HspA1A in MSC under low-oxygen conditions, suppressed the expression of a Bax protein, and inhibited the phosphorylation (i.e., activation) of JNK (c-Jun N-terminal kinase, stress activated protein kinase) while maintaining the expression level thereof, thus suppressing apoptosis.

FIGS. 3A-F are pictures of retinal cells in a retinal degeneration model. In a retinal degeneration model having apoptosis induced by the anticancer agent MNU, the degeneration of the photoreceptor cell layer occurred starting from the central portion of the retina (FIG. 3A). Unlike the control group, the central portion of the retina showed a decrease in the cells of the photoreceptor cell layer and was changed into an irregular shape. At the middle portion of the retina, the cell layer was better maintained than in a photograph of the central portion with little damage to the cells (FIG. 3B). The peripheral portion of the retina almost completely maintained its appearance (FIG. 3C).

FIGS. 3D-F show the conjunctiva after administration of PTD-HspA1A. The central portion of the retina showed serious damage to the photoreceptor cell layer, but was conserved at a portion thereof, showing that the PTD-HspA1A had an effect, as compared to the control group (FIG. 3D). In the middle portion of the retina, the photoreceptor cells were better conserved as the normal photoreceptor cells could be more clearly observed than in the photograph of the central portion of the retina (FIG. 3E). At the peripheral portion of the retina, the photoreceptor cell layer was conserved to an extent almost equal to the case of the systemic administration (FIG. 3F). The peripheral portion was morphologically virtually normal.

FIG. 4 shows the normal maintenance of intestinal epithelial cells with HspA1A expressed. Isolated intestinal epithelial cells were divided into two groups, only one of which was given heat shock at 43° C. to induce the expression of the HspA1A protein. Cells having the HspA1A protein expressed therein were normally maintained (left photograph), whereas the cells of the group without HspA1A protein expressed therein exhibited condensation of the nucleus and cytoplasm (right photograph).

FIG. 5 shows the comparison of the DAPI-labeled MSCs treated with and without Hph-1-HspA1A in the host myocardium.

FIGS. 6A-C show the analysis of myocardial repair after the implantation of the HspA1A-MSC into the infarcted myocardium. (FIG. 6A) H&E and DAPI double staining show that the viable, mature cardiac myocytes have infiltrated into the scar area by 4 weeks after the implantation. (FIG. 6B) Double staining of DAPI and the cardiac specific markers, CTn T, MHC, or Cav2.1, show that the cardiac specific markers are expressed in the DAPI-labeled cells. The cardiac specific markers are indicated in red. (FIG. 6C) Double staining of DAPI and connexin-43 or N-cadherin show that the MSC-derived cardiac myocytes express connexin-43 and N-cadherin at the border zone of the implanted cells and the host myocytes. Connexin-43 and N-cadherin staining is shown in green.

FIGS. 7A-B show the apoptosis-suppressing effect of Hsc70. (FIG. 7A) Purification of Hsc70. (FIG. 7B) Apoptosis-suppressing effect of Hsc70 in a concentration dependent manner. Control represents Jurkat T cells without any treatment, and STS represents staurosporin treatment.

FIGS. 8A-B show the apoptosis-suppressing effect of cvHsp. (FIG. 8A) Purification of cvHsp. (FIG. 8B) Apoptosis-suppressing effect of cvHsp in a concentration dependent manner.

FIG. 9 shows a Table with members of the human Hsp70 family.

### DETAILED DESCRIPTION OF THE INVENTION

Apoptotic and necrotic cell death, and other programmed cell death pathways are often involved in ischemic brain injury, heart disease, and neurodegenerative disease. The present invention encompasses methods for treating or preventing apoptotic cell death using a conjugate or fusion of a peptide protein transduction domain (PTD) and a heat-shock protein (Hsp). The inventive conjugate can be prepared by fusing a PTD-encoding gene with an Hsp gene by cloning. The PTDs used in the present invention are capable of delivering proteins, peptides and chemical compounds into the body through the skin, eyeball or airway, and thus, if provided as a conjugate with a polypeptide, can deliver the polypeptide to a topical area in vivo.

One embodiment of the present invention is the use of a PTD-Hsp70 conjugate to treat or prevent apoptotic cell death. According to the present invention, the Hsp70 easily passes through the cellular membrane due to the intracellular penetration and delivery effects of PTD and is delivered into cells. The conjugate delivered into the cells is decomposed by intracellular proteases and, as a result, the separated Hsp70 shows the effects of inhibiting and treating diseases and suppressing apoptosis.

Another embodiment of the present invention is a method of reducing or inhibiting apoptosis of a cell population whereby a cell population is contacted with an effective amount of PTD-Hsp such that one or more cells that are subject to apoptosis are protected from cell death. The cells can be differentiated cells or precursor cells and include, but are not limited to, neural cells (e.g., neurons), fibroblasts, smooth muscle cells, tumor cells, haematopoietic cells, monocytes, macrophages, epithelial cells, keratinocytes, nerve cells, endothelial cells, granulocytes, monocytes, erythrocytes, lymphocytes and platelets. The term “contacting” as used herein means exposing the cells to PTD-Hsp thereby inhibiting apoptosis in the cells and allowing the cells to proliferate and accumulate. The cells can be contacted with PTD-Hsp ex vivo or in vivo.

Another embodiment of the present invention is a method of treating, preventing or suppressing a pathological condition characterized by an elevated level of apoptosis, by administering to an individual in need of such treatment an amount of PTD-Hsp effective for treating the pathological condition. The pathological conditions contemplated include, but are not limited to, stress-induced pathologies, such as ischemia, and chronic degenerative diseases, such as neurodegenerative diseases and degenerative atrophy.

Ischemic conditions include, but are not limited to, stroke due to ischemic cerebral infarction, ischemic acute renal failure, intestinal ischemia, ischemic heart disease due to myocardial infarction (myocardial ischemia and disorder after reperfusion, liver ischemia, brain ischemia (e.g., brain ischemia from apoplexy and the like) and ischemia retinae.

Neurodegenerative diseases include, but are not limited to, Alzheimer's disease, Parkinson's disease, Huntington's disease, multiple sclerosis, amyotrophic lateral sclerosis (ALS), spinobulbar atrophy, denervation atrophy, spinal muscular dystrophy (SMA), pigmentary degeneration of the retina and glaucoma, cerebellar degeneration and neonatal jaundice, otosclerosis, stroke, dementia, and successive delayed neuronal death (DND).

Additional degenerative diseases of the heart include, but are not limited to, myasthenia gravis, viral myocarditis, autoimmune myocarditis (congestive cardiomyopathy and chronic myocarditis), myocardial disorders or death due to hypertrophic heart and heart failure, arrythmogenic right ventricular cardiomyopathy, heart failure, and coronary artery by-pass graft.

Other degenerative diseases include, alcoholic hepatitis, viral hepatitis, renal diseases (e.g., glomerulonephritis), hemolytic uremic symdrome and the like, acquired immunodeficiency syndrome (AIDS), inflammatory skin disorders such as toxic epidermal necrolysis (TEN) and multiform exudative erythema, graft versus host disease (GVH), radiation disorders, side effects due to anti-cancer drugs, anti-viral drugs and the like, disorders due to toxic agents such as sodium azide, potassium cyanide and the like, osteomyelo-dysplasia such as aplastic anemia and the like, prion diseases such as Creutzfeldt-Jakob's disease, spinal cord injury, traumatic brain injury, cytotoxic T cell or natural killer cell-mediated apoptosis associated with autoimmune disease and transplant rejection, mitochondrial drug toxicity, e.g., as a result of chemotherapy or HIV therapy, viral, bacterial, or protozoal infection, inflammation or inflammatory diseases, inflammatory bowel disease, sepsis and septic shock, follicule to ovocyte stages, from ovocyte to mature egg stages and sperm (e.g., methods of freezing and transplanting ovarian tissue, artificial fecondation), skin damage (due to exposure to high level of radiation, heat, burns, chemicals, sun, and autoimmune diseases), myelodysplastic syndromes (MDS) (death of bone marrow cells), pancreatitis, osteoarthritis, rheumatoid arthritis, psoriasis, glomerulonephritis, atherosclerosis, and graft versus host disease, retinal pericyte apopotosis, retinal neurons apoptosis glaucoma, retinal damages resulting from ischemia, diabetic retinopathy, respiratory syndrome, diabetes (e.g., insulin dependent diabetes), autoimmune disease, acquired poly glutamine disease, Monckeberg's, encephalopathy associated with acquired immunodeficiency disease (AIDS), myopathies and muscular dystrophies, glomerulosclerosis, Monckeberg's medial sclerosis, inflammatory bowel disease, Crohn's disease, autoimmune hepatitis, hemochromatosis and Wilson disease, alcoholic hepatitis, acute hepatic failure of different etiology, diseases of the bile ducts, atherosclerosis, hypertension, apoptosis-induced hair loss and apoptosis associated with the use of chemotherapeutic drugs.

Another embodiment of the invention is a method for expanding or increasing survival of a cell population by contacting the cells with an inhibiting and/or suppressing amount of PTD-Hsp, which suppresses apoptosis in the cell population, thereby expanding or increasing survival of the cell population. The term “expanding” as used herein means increasing the number of cells of a pre-existing cell population. The term “survival” refers to maintaining viability of cells, typically ex vivo; however, the term is meant to include in vivo as well. Survival may be from a few hours to several days or longer. The cell population can consist of differentiated cells or precursor cells, granulocytes, monocytes, erythrocytes, lymphocytes or platelets.

The method includes contacting the desired cells with an effective amount of PTD-Hsp, which inhibits or suppresses apoptosis in the cell population. The term “contacting” as used herein means exposing the cells to PTD-Hsp thereby inhibiting apoptosis in the cells and allowing the cells to proliferate and accumulate. The cells can be contacted with PTD-Hsp ex vivo or in vivo.

An additional embodiment of the invention is a method of regenerating damaged cells, comprising storing the cells in an effective amount of a solution comprising PTD-Hsp, whereby damaged cells are regenerated.

A further embodiment of the invention is a method for prolonging cell, tissue or organ viability comprising contacting a cell population, tissue or organ with an amount of PTD-Hsp effective to suppress apoptosis in one or more cells of the cell population, tissue or organ, thereby prolonging the viability of the cell population, tissue or organ as compared to an untreated cell population, tissue or organ. The cells in the cell population can be damaged cells, whereby contact with PTD-Hsp results in regeneration of the damaged cells. The treated cells, tissues, and organs may be used, inter alia, for transfusions or transplantation. The cell population, tissue or organ can be contacted with PTD-Hsp during transfusions or during transplantation of the cell population, tissue or organ.

The cells can be differentiated cells or precursor cells and include, but are not limited to, stem cells (e.g., hematopoietic, mesenchymal, stromal or neural stem cells), neural or nerve cells (e.g., neurons), fibroblasts, smooth muscle cells, tumor cells, hematopoietic cells, monocytes, macrophages, epithelial cells, keratinocytes, endothelial cells, granulocytes, erythrocytes, lymphocytes and platelets.

The hematopoietic stem cells can be transplanted into an individual in need thereof and are capable of differentiating into blood cells. The individual can be a leukemia or blood cancer patient.

Mesenchymal stem cells (MSCs) are cells which are capable of differentiating into more than one type of mesenchymal cell lineage. MSCs have been identified and cultured from avian and mammalian species including mouse, rat, rabbit, dog and human (see U.S. Pat. No. 5,486,359). Isolation, purification and culture expansion of human MSCs is described in detail therein. MSCs can be transplanted into an individual in need thereof and are capable of differentiating into bone cells (e.g., osteocytes), cartilage cells (e.g., chondrocytes), fat cells (e.g., adipocytes), or cardiomyocytes. MSCs can be transplanted into a heart, including an infarcted heart.

The neural stem cells can be transplanted into an individual in need thereof and are capable of differentiating into nerve cells such as neurons or non-nerve cells, such as astrocytes or oligodendrocytes.

In addition, the cell population, tissue or organ can be contacted with PTD-Hsp to inhibit apoptosis, thereby increasing cell viability during bioproduction. By enhancing bioproduction, cells survive longer and produce and/or secrete a desired product longer, thus resulting in a greater yield of product. The ability to prevent apoptosis may allow cells to live independent of normal required growth factors, reducing the cost of media supplements.

The term “contacting” as used herein means exposing the cells to PTD-Hsp thereby inhibiting apoptosis in the cells and allowing the cells to proliferate and accumulate. The cells can be contacted with PTD-Hsp ex vivo or in vivo.

The term “prolonging” means that a tissue or organ for transplantation is preserved by treatment using the method of the invention as compared to a similar tissue or organ that has not been treated with PTD-Hsp. It is believed that contacting the cells or organ for transplantation with PTD-Hsp inhibits apoptosis, thereby preserving the organ and prolonging viability.

The cell population, tissue or organ may be contacted with the PTD-Hsp ex vivo or in vivo during transfusions or transplantation such that damage caused by reperfusion of the organ or tissue is decreased or prevented. The contacting can occur by administering to a tissue or organ donor PTD-Hsp prior to, or concurrent with, removal of the cell population, tissue or organ. The organ can be any solid organ including, but not limited to, the heart, pancreas, kidney, lung, or liver.

The PTD-Hsp may be in a solution, such as a hypothermic storage solution, and storage may occur at temperatures above the freezing point or below the freezing point. The basic challenge of hypothermic storage is to preserve the material in a state that can be reversed without causing extensive cell damage or cell death. The solution may further comprise an amount of a vitrification composition effective to prevent the formation of ice crystals both in the solution, and the cell, tissue or organ. U.S. Pat. No. 6,045,990, incorporated herein by reference, demonstrates in part that survival and recovery from cryopreservation can be enhanced by the inclusion of anti-apoptotic agents in the preservation solution or medium.

The method comprises: a) contacting the cell, tissue or organ with a hypothermic storage solution, wherein the solution comprises: i) a composition that inhibits apoptosis; and ii) a concentration of a vitrification composition that is sufficient for vitrification of the solution; and b) vitrifying the cell, tissue or organ, wherein the vitrification occurs both within the cell, tissue or organ and in the hypothermic storage solution comprising and comprised by the cell, tissue or organ.

The vitrification is accomplished through use of a hypothermic storage solution comprising an agent that prevents ice nucleation within the extracellular and intracellular environment thereby preventing ice formation and that has a glass transition temperature (Tg) lower than the homogeneous nucleation temperature of the solution. Reduction of the temperature of a sample in a hypothermic storage solution to below the glass transition temperature results in vitrification of the solution and the cell, tissue or organ in that solution. Under these circumstances, there is no crystalline ice formation in or around the cells as the sample becomes a solid. The inclusion of one or more anti-apoptotic agents aids in preventing the apoptotic cell death that normally occurs following this type of preservation.

In yet another embodiment, the invention provides a method for increasing survival of cells cultured in vitro for utilities other than transplantation. Cell death during fermentation has been shown to be apoptotic, thus inhibition of apoptosis will increase cell viability during bioproduction. Inhibition of apoptosis is of use in enhancing bioproduction in vitro whereby host cells that produce a product of interest are contacted with PTD-Hsp, wherein PTD-Hsp suppresses apoptosis in one or more cells, thereby increasing survival of the cells in vitro. By enhancing bioproduction, cells survive longer and produce and/or secrete a desired product longer, thus resulting in a greater yield of product. The ability to prevent apoptosis may allow cells to live independent of normal required growth factors, reducing the cost of media supplements.

Effectiveness of PTD-Hsp on bioproduction can be measured in several ways: 1) determining the percentage of apoptotic cells in the culture at different time points; 2) determining the useful lifespan of the culture with regards to production of the desired product; 3) measuring the yield of product per gram of cells or per volume of culture; or 4) measuring final purity of the product.

### Protein Transduction Domain (PTD)

The PTD effectively allows delivery or uptake of proteins, peptides and chemical compounds of interest in vivo and in vitro into cells by systemic or local administration. Administration routes include routes that are, inter alia, intramuscular, intraperitoneal, intravenous, oral, nasal, subcutaneous, intradermal, mucosal, and by inhalation. Thus, if the PTD is provided as a conjugate with a protein, peptide and/or chemical compound, the PTD can deliver the protein, peptide and/or chemical compound to a topical area, e.g., skin, eyeball or airway.

The present inventors compared various PTDs with each other and, as a result, found that the PTDs contain a relatively large number of lysines and arginines, and particularly arginine is important in the delivery of substances into cells. This was supported by the fact that artificial peptides consisting of positively charged amino acids also have the effect of delivering substances (see Laus, R., et al., Nature Biotechnol. 18:1269-1272 (2000)).

It was found that certain proteins were delivered into cells by 9-12 arginine residues or 9-12 lysine residues (see Rothbard, J. B., et al., Nature Med. 6:1253-1257 (2000)) in contradiction to the hypothesis that the arginine residues or lysine residues are present at certain locations in the PTD itself to form a channel structure. It was also found that only target proteins covalently or non-covalently bonded with the PTD are delivered into cells, contradicting the hypothesis that the PTD destroys the cell membrane to deliver macromolecules into cells. Our study results demonstrated that the delivery of substances into cells by the PTD effectively occurs at both 37° C. and 4° C.

MTS (Membrane Translocating Sequence) is a new PTD having characteristics different from those of the above existing PTDs. MTS was synthesized and constructed based on the amino acid sequence of a signal peptide of FGF (fibroblast growth factor) (see Jo, D., et al., Nat. Biotechnol. 19:929-933 (2001)). However, the amino acid sequence of the signal peptide has the following characteristics which are significantly different from those of the above existing PTD amino acids: (a) 3-5 arginine or lysine residues are discontinuously present, like serine or threonine residues, and glutamic acid or asparaginic acid is not present; (b) at least one basic amino acid and 6-12 hydrophobic amino acids are present; (c) serine, threonine and small-sized hydrophobic amino acids are large in number and glutamic acid and asparaginic acid are small in number; (d) the C-terminal portion contains a large number of serine, lysine and leucine residues; and (e) one or two basic amino acids are clustered together, and 10 random amino acids are present between these basic amino acids. That is to say, PTDs such as MTS do not have the characteristics of the amino acid constitution of the existing PTDs.

The present inventors have found that unfolded proteins are much more effectively delivered than proteins having a complex three-dimensional structure, and that unfolded proteins are not released out of cells or extracellular organelles, after they are delivered into cells and intracellular organelles. In addition, PTDs do not utilize endocytosis or phagocytosis with receptors, but may use channels present on the cell surface. Thus, hydrophobic amino acids such as alanine and valine should be present in the PTD.

The present inventors have found that, if the peptide consisting of amino acid residues 858-868 of human transcription factor Hph-1 is used as a peptide for delivering substances into cells, it can deliver target proteins, nucleic acids, fats, carbohydrates or chemical compounds in vivo or in vitro into the cytoplasm, organelle or nucleus of eukaryotic or prokaryotic cells, thereby completing the present invention.

For use as the PTD in the present invention, the present inventors constructed several peptides using a solid synthesis method, but it is to be understood that other kinds of PTD can be used depending on the desired delivery area and the kind of linker used. The PTD consists of 3-30 amino acids, more preferably 5-15 amino acids, at least 30% of which are preferably arginine residues. However, PTDs without any arginine residues are also contemplated.

One embodiment involves the use of Hph-1-PTD, the PTD from the human (and mouse) transcription factor HPH-1 (YARVRRRGPRR) (SEQ ID NO:1). Another embodiment involves the use of the PTD of Sim-2 (AKAARQAAR) (SEQ ID NO:2).

Other embodiments include, but are not limited to, the PTDs of HIV-1 viral protein Tat (YGRKKRRQRRR) (SEQ ID NO:3), Antennapedia protein (Antp) of Drosophila (RQIKIWFQNRRMKWKK) (SEQ ID NO:4), HSV-1 structural protein Vp22 (DAATATRGRSAASRPTERPRAPARSASRPRRPVE) (SEQ ID NO:5), regulator of G protein signaling R7 (RRRRRRR) (SEQ ID NO:6), MTS (AAVALLPAVLLALLAPAAADQNQLMP) (SEQ ID NO:7), and short amphipathic peptide carriers Pep-1 (KETWWETWWTEWSQPKKKRKV) (SEQ ID NO:8) and Pep-2 (KETWFETWFTEWSQPKKKRKV) (SEQ ID NO:9).

### Heat Shock Protein (Hsp)

To achieve the above objects, the present invention provides a conjugate of a PTD and a polypeptide, such as an Hsp chaperone, co-chaperone, or low molecular weight heat shock or small stress protein (smHsp).

The Hsps are classified into about six families, including the Hsp10, Hsp40, Hsp60, Hsp70, Hsp90 and Hsp100 families, on the basis of their monomeric molecular weight. Hsp40 is a co-chaperone for Hsp70 activities (Van Eden, W., et al., Nat. Rev. Immunol. 5:318-330 (2005)). Hsp families are highly conserved and some mammalian family members have highly conserved microbial homologues, which results in immunological cross-recognition between mammalian and microbial homologues (Van Eden, W., et al., Nat. Rev. Immunol. 5:318-330 (2005)).

The smHsp family of proteins have been shown to play a role in stabilizing protein folding and transport chiefly through the modulation of actin polymerization and cytoskeletal organization. The presence of an evolutionarily conserved α-crystalline domain at the C-terminus of about 80-100 residues characterizes all smHsps. This domain is preceded by an N-terminal domain, which is variable in size and sequence, and is followed by a short, poorly conserved C-terminal extension, known to undergo numerous modifications including truncations. Examples of smHsps include cvHsp, αB-crystallin, αA-crystallin, Hsp20, Hsp P-2, Hsp-like 27 and Hsp27 (see Krief et al., J. Biol. Chem. 274:36592-36600 (1999)).

One embodiment of the present invention is the conjugate of a PTD with a member of the Hsp70 family of proteins. The Hsp70 family of proteins have been shown to suppress multiple types of cell death, including necrotic cell death, classical apoptosis, and other programmed cell death pathways that are caspase-independent and not blocked by Bcl-2 (see Giffard, R. G., et al., J. Exp. Biol. 207:3213-3220 (2004)). The members of the Hsp70 family of proteins include, but are not limited to, HspA1A, HspA1B, HspA1L, HspA2A, HspA2B, HspA4, HspA5, HspA6, HspA7, Hsp8A (Hsc70), Hsp9A, and are also contemplated. The amino acid sequences of these members of the Hsp70 family of proteins are provided below.

```
            The nucleotide sequence of HspA1A (SEQ ID NO: 10) is:
            atggccaaagccgcggcgatcggcatcgacctgggcaccacctactcctgcgtgggggtgttccaacacggcaag
gtggagatcatcgccaacgaccagggcaaccgcaccacccccagctacgtggccttcacggacaccgagcggctcatcggg
gatgcggccaagaaccaggtggcgctgaacccgcagaacaccgtgtttgacgcgaagcggctgatcggccgcaagttcggc
gacccggtggtgcagtcggacatgaagcactggcctttccaggtgatcaacgacggagacaagcccaaggtgcaggtgagct
acaagggggacaccaaggcattctaccccgaggagatctcgtccatggtgctgaccaagatgaaggagatcgccgaggcgta
cctgggctacccggtgaccaacgcggtgatcaccgtgccggcctacttcaacgactcgcagcgccaggccaccaaggatgcg
ggtgtgatcgcggggctcaacgtgctgcggatcatcaacgagcccacggccgccgccatcgcctacggcctggacagaacg
ggcaagggggagcgcaacgtgctcatctttgacctgggcgggggcaccttcgacgtgtccatcctgacgatcgacgacggcat
cttcgaggtgaaggccacggccggggacacccacctgggtggggaggactttgacaacaggctggtgaaccacttcgtggag
gagttcaagagaaaacacaagaaggacatcagccagaacaagcgagccgtgaggcggctgcgcaccgcctgcgagagggc
caagaggaccctgtcgtccagcacccaggccagcctggagatcgactccctgtttgagggcatcgacttctacacgtccatcac
cagggcgaggttcgaggagctgtgctccgacctgttccgaagcaccctggagcccgtggagaaggctctgcgcgacgccaag
ctggacaaggcccagattcacgacctggtcctggtcgggggctccacccgcatccccaaggtgcagaagctgctgcaggactt
cttcaacgggcgcgacctgaacaagagcatcaaccccgacgaggctgtggcctacggggcggcggtgcaggcggccatcct
gatgggggacaagtccgagaacgtgcaggacctgctgctgctggacgtggctcccctgtcgctggggctggagacggccgg
aggcgtgatgactgccctgatcaagcgcaactccaccatccccaccaagcagacgcagatcttcaccacctactccgacaacca
acccggggtgctgatccaggtgtacgagggcgagagggccatgacgaaagacaacaatctgttggggcgcttcgagctgagc
ggcatccctccggcccccaggggcgtgccccagatcgaggtgaccttcgacatcgatgccaacggcatcctgaacgtcacgg
ccacggacaagagcaccggcaaggccaacaagatcaccatcaccaacgacaagggccgcctgagcaaggaggagatcga
gcgcatggtgcaggaggcggagaagtacaaagcggaggacgaggtgcagcgcgagagggtgtcagccaagaacgccctg
gagtcctacgccttcaacatgaagagcgccgtggaggatgaggggctcaagggcaagatcagcgaggccgacaagaagaag
gtgctggacaagtgtcaagaggtcatctcgtggctggacgccaacaccttggccgagaaggacgagtttgagcacaagaggaa
ggagctggagcaggtgtgtaaccccatcatcagcggactgtaccagggtgccggtggtcccgggcctgggggcttcggggct
cagggtcccaagggagggtctgggtcaggccccaccattgaggaggtagattag
            The amino acid sequence of HspA1A (SEQ ID NO: 11) is:
            MAKAAAIGIDLGTTYSCVGVFQHGKVEIIANDQGNRTTPSYVAFTDTERLI
GDAAKNQVALNPQNTVFDAKRLIGRKFGDPVVQSDMKHWPFQVINDGDKPKVQ
VSYKGDTKAFYPEEISSMVLTKMKEIAEAYLGYPVTNAVITVPAYFNDSQRQATK
DAGVIAGLNVLRIINEPTAAAIAYGLDRTGKGERNVLIFDLGGGTFDVSILTIDDGI
FEVKATAGDTHLGGEDFDNRLVNHFVEEFKRKHKKDISQNKRAVRRLRTACERA
KRTLSSSTQASLEIDSLFEGIDFYTSITRARFEELCSDLFRSTLEPVEKALRDAKLDK
AQIHDLVLVGGSTRIPKVQKLLQDFFNGRDLNKSINPDEAVAYGAAVQAAILMG
DKSENVQDLLLLDVAPLSLGLETAGGVMTALIKRNSTIPTKQTQIFTTYSDNQPG
VLIQVYEGERAMTKDNNLLGRFELSGIPPAPRGVPQIEVTFDIDANGILNVTATDK
STGKANKITITNDKGRLSKEEIERMVQEAEKYKAEDEVQRERVSAKNALESYAFN
MKSAVEDEGLKGKISEADKKKVLDKCQEVISWLDANTLAEKDEFEHKRKELEQ
VCNPIISGLYQGAGGPGPGGFGAQGPKGGSGSGPTIEEVD
            The amino acid sequence of HspA1B (SEQ ID NO: 12) is:
            MAKAAAIGIDLGTTYSCVGVFQHGKVEIIANDQGNRTTPSYVAFTDTERLI
GDAAKNQVALNPQNTVFDAKRLIGRKFGDPVVQSDMKHWPFQVINDGDKPKVQ
VSYKGETKAFYPEEISSMVLTKMKEIAEAYLGYPVTNAVITVPAYFNDSQRQATK
DAGVIAGLNVLRIINEPTAAAIAYGLDRTGKGERNVLIFDLGGGTFDVSILTIDDGI
FEVKATAGDTHLGGEDFDNRLVNHFVEEFKRKHKKDISQNKRAVRRLRTACERA
KRTLSSSTQASLEIDSLFEGIDFYTSITRARFEELCSDLFRSTLEPVEKALRDAKLDK
AQIHDLVLVGGSTRIPKVQKLLQDFFNGRDLNKSINPDEAVAYGAAVQAAILMG
DKSENVQDLLLLDVAPLSLGLETAGGVMTALIKRNSTIPTKQTQIFTTYSDNQPG
VLIQVYEGERAMTKDNNLLGRFELSGIPPAPRGVPQIEVTFDIDANGILNVTATDK
STGKASKITITNDKGRLSKEEIERMVQEAEKYKAEDEVQRERVSAKNALESYAFN
MKSAVEDEGLKGKISEADKKKVLDKCQEVISWLDANTLAEKDEFEHKRKELEQ
VCNPIISGLYQGAGGPGPGGFGAQGPKGGSGSGPTIEEVD
            The amino acid sequence of HspA1L (SEQ ID NO: 13) is:
            MATAKGIAIGIDLGTTYSCVGVFQHGKVEIIANDQGNRTTPSYVAFTDTER
LIGDAAKNQVAMNPQNTVFDAKRLIGRKFNDPVVQADMKLWPFQVINEGGKPK
VLVSYKGENKAFYPEEISSMVLTKLKETAEAFLGHPVTNAVITVPAYFNDSQRQA
TKDAGVIAGLNVLRIINEPTAAAIAYGLDKGGQGERHVLIFDLGGGTFDVSILTID
DGIFEVKATAGDTHLGGEDFDNRLVSHFVEEFKRKHKKDISQNKRAVRRLRTAC
ERAKRTLSSSTQANLEIDSLYEGIDFYTSITRARFEELCADLFRGTLEPVEKALRDA
KMDKAKIHDIVLVGGSTRIPKVQRLLQDYFNGRDLNKSINPDEAVAYGAAVQAA
ILMGDKSEKVQDLLLLDVAPLSLGLETAGGVMTALIKRNSTIPTKQTQIFTTYSDN
QPGVLIQVYEGERAMTKDNNLLGRFDLTGIPPAPRGVPQIEVTFDIDANGILNVTA
MDKSTGKVNKITITNDKGRLSKEEIERMVLDAEKYKAEDEVQREKIAAKNALES
YAFNMKSVVSDEGLKGKISESDKNKILDKCNELLSWLEVNQLAEKDEFDHKRKE
LEQMCNPIITKLYQGGCTGPACGTGYVPGRPATGPTIEEVD
            The amino acid sequence of HspA2A (SEQ ID NO: 14) is:
            MSARGPAIGIDLGTTYSCVGVFQHGKVEIIANDQGNRTTPSYVAFTDTERL
IGDAAKNQVAMNPTNTIFDAKRLIGRKFEDATVQSDMKHWPFRVVSEGGKPKV
QVEYKGETKTFFPEEISSMVLTKMKEIAEAYLGGKVHSAVITVPAYFNDSQRQAT
KDAGTITGLNVLRIINEPTAAAIAYGLDKKGCAGGEKNVLIFDLGGGTFDVSILTIE
DGIFEVKSTAGDTHLGGEDFDNRMVSHLAEEFKRKHKKDIGPNKRAVRRLRTAC
ERAKRTLSSSTQASIEIDSLYEGVDFYTSITRARFEELNADLFRGTLEPVEKALRDA
KLDKGQIQEIVLVGGSTRIPKIQKLLQDFFNGKELNKSINPDEAVAYGAAVQAAIL
IGDKSENVQDLLLLDVTPLSLGIETAGGVMTPLIKRNTTIPTKQTQTFTTYSDNQSS
VLVQVYEGERAMTKDNNLLGKFDLTGIPPAPRGVPQIEVTFDIDANGILNVTAAD
KSTGKENKITITNDKGRLSKDDIDRMVQEAERYKSEDEANRDRVAAKNALESYT
YNIKQTVEDEKLRGKISEQDKNKILDKCQEVINWLDRNQMAEKDEYEHKQKELE
RVCNPIISKLYQGGPGGGSGGGGSGASGGPTIEEVD
            The amino acid sequence of HspA2B (SEQ ID NO: 15) is:
            MSARGPAIGIDLGTTYSCVGVFQHGKVEIIANDQGNRTTPSYVAFTDTERL
IGDAAKNQVAMNPTNTIFDAKRLIGRKFEDATVQSDMKHWPFRVVSEGGKPKV
QVEYKGETKTFFPEEISSMVLTKMKEIAEAYLGGKVHSAVITVPAYFNPSQRQAT
KDAGTITGLNVLRIINEPTAAAIAYGLDKKGCAGGEKNVLIFDLGGGTFDVSILTIE
DGIFEVKSTAGDTHLGGEDFDNRMVSHLAEEFKRKHKKDIGPNKRAVRRLRTAC
ERAKRTLSSSTQASIEIDSLYEGVDFYTSITRARFEELNADLFRGTLEPVEKALRDA
KLDKGQIQEIVLVGGSTRIPKIQKLLQDFFNGKELNKSINPDEAVAYGAAVQAAIL
IGDKSENVQDLLLLDVTPLSLGIETAGGVMTPLIKRNTTIPTKQTQTFTTYSDNQSS
VLVQVYEGERAMTKDNNLLGKFDLTGIPPAPRGVPQIEVTFDIDANGILNVTAAD
KSTGKENKITITNDKGRLSKDDIDRMVQEAERYKSEDEANRDRVAAKNALESYT
YNIKQTVEDEKLRGKISEQDKNKILDKCQEVINWLDRNQMAEKDEYEHKQKELE
RVCNPIISKLYQGGPGGGSGGGGSGASGGPTIEEVD
            The amino acid sequence of HspA4 (SEQ ID NO: 16) is:
            MSVVGIDLGFQSCYVAVARAGGIETIANEYSDRCTPACISFGPKNRSIGAA
AKSQVISNAKNTVQGFKRFHGRAFSDPFVEAEKSNLAYDIVQLPTGLTGIKVTYM
EEERNFTTEQVTAMLLSKLKETAESVLKKPVVDCVVSVPCFYTDAERRSVMDAT
QIAGLNCLRLMNETTAVALAYGIYKQDLPALEEKPRNVVFVDMGHSAYQVSVC
AFNRGKLKVLATAFDTTLGGRKFDEVLVNHFCEEFGKKYKLDIKSKIRALLRLSQ
ECEKLKKLMSANASDLPLSIECFMNDVDVSGTMNRGKFLEMCNDLLARVEPPLR
SVLEQTKLKKEDIYAVEIVGGATRIPAVKEKISKFFGKELSTTLNADEAVTRGCAL
QCAILSPAFKVREFSITDVVPYPISLRWNSPAEEGSSDCEVFSKNHAAPFSKVLTFY
RKEPFTLEAYYSSPQDLPYPDPAIAQFSVQKVTPQSDGSSSKVKVKVRVNVHGIFS
VSSASLVEVHKSEENEEPMETDQNAKEEEKMQVDQEEPHVEEQQQQTPAENKA
ESEEMETSQAGSKDKKMDQPPQAKKAKVKTSTVDLPIENQLLWQIDREMLNLYI
ENEGKMIMQDKLEKERNPAKNAVREYVYEMRDKLSGEYEKFVSEDGRNSFTLK
LEDTENWLYEDGEDQPKQVYVDKLAELKNLGQPIKIRFQESEERPKLFEELGKQI
QQYMKIISSFKNKEDQYDHLDAADMTKVEKSTNEAMEWMNKLNLQNKQSLT
MDPVVKSKEIEAKIKELTSTCSPIISKPKPKVEPPKEEQKNAEQNGPVDGQGDNPG
PQAAEQGTDTAVPSDSDKXLPEMDID
            The amino acid sequence of HspA5 (SEQ ID NO: 17) is:
            MKLSLVAAMLLLLSAARAEEEDKKEDVGTVVGIDLGTTYSCVGVFKNGR
VEIIANDQGNPITPSYVAFTPEGERLIGDAAKNQLTSNPENTVFDAKRLIGRTWND
PSVQQDIKFLPFKVVEKKTKPYIQVDIGGGQTKTFAPEEISAMVLTKMKETAEAY
LGKKVTHAVVTVPAYFNDAQRQATKDAGTIAGLNVMRIINEPTAAAIAYGLDKR
EGEKNILVFDLGGGTFDVSLLTIDNGVFEVVATNGDTHLGGEDFDQRVMEHFIKL
YKKKTGRDVRKDNRAVQKLRREVEKAKRALSSQHQARIEIESFYEGEDFSETLTR
AKFEELNMDLFRSTMKPVQKVLEDSDLKKSDIDEIVLVGGSTRIPKIQQLVKEFFN
GKEPSRGINPDEAVAYGAAVQAGVLSGDQDTGDLVLLDVCPLTLGIETVGGVMT
KLIPRNTVVPTKKSQIFSTASDNQPTVTIKVYEGERPLTKDNHLLGTFDLTGIPPAP
RGVPQIEVTFEIDVNGILRVTAEDKGTGNKNKITITNDQNRLTPEEIERMVNDAEK
FAEEDKKLKERIDTRNELESYAYSLKNQIGDKEKLGGKLSSEDKETMEKAVEEKI
EWLESHQDADIEDFKAKKKELEEIVQPIISKLYGSAGPPPTGEEDTAEKDEL
            The amino acid sequence of HspA6 (SEQ ID NO: 18) is:
            MQAPRELAVGIDLGTTYSCVGVFQQGRVEILANDQGNRTTPSYVAFTDTE
RLVGDAAKSQAALNPHNTVFDAKRLIGRKFADTTVQSDMKHWPFRVVSEGGKP
KVRVCYRGEDKTFYPEEISSMVLSKMKETAEAYLGQPVKHAVITVPAYFNDSQR
QATKDAGAIAGLNVLRIINEPTAAAIAYGLDRRGAGERNVLIFDLGGGTFDVSVL
SIDAGVFEVKATAGDTHLGGEDFDNRLVNHFMEEFRRKHGKDLSGNKRALRRL
RTACERAKRTLSSSTQATLEIDSLFEGVDFYTSITRARFEELCSDLFRSTLEPVEKA
LRDAKLDKAQIHDVVLVGGSTRIPKVQKLLQDFFNGKELNKSINPDEAVAYGAA
VQAAVLMGDKCEKVQDLLLLDVAPLSLGLETAGGVMTTLIQRNATIPTKQTQTF
TTYSDNQPGVFIQVYEGERAMTKDNNLLGRFELSGIPPAPRGVPQIEVTFDIDANG
ILSVTATDRSTGKANKITITNDKGRLSKEEVERMVHEAEQYKAEDEAQRDRVAA
KNSLEAHVFHVKGSLQEESLRDKIPEEDRRKMQDKCREVLAWLEHNQLAEKEEY
EHQKRELEQICRPIFSRLYGGPGVPGGSSCGTQARQGDPSTGPIIEEVD
            The amino acid sequence of HspA7 (SEQ ID NO: 19) is:
            MQAPRELAVGIDLGTTYSCVGVFQQGRVEILANDQGNRTTPSYVAFTDTE
RLVGDAAKNQAALNPHNTVFDAKRLIGRKFADTTVQSDMKHWPFKVVSGGGKP
KVRVCYRGEDKTFYPEEISSMVLTKMKETAEAYLGQPVKHAVITVPTYFSNSQR
QATKDAGAIAGLKVLPIINEATAAAIAYGLDRRRAGKRNVLIFDLGGGTFDVSVL
TIDAGVFEVKATAGDTHLGGEDFDNRLVNHFMEEF
            The amino acid sequence of Hsp8A (HSC70) (SEQ ID NO: 20) is:
            MSKGPAVGIDLGTTYSCVGVFQHGKVEIIANDQGNRTTPSYVAFTDTERLI
GDAAKNQVAMNPTNTVFDAKLIGRRFDDAVVQSDMKHWPFMVVNDAGRPK
VQVEYKGETKSFYPEEVSSMVLTKMKEIAEAYLGKTVTNAVVTVPAYFNDSQRQ
ATKDAGTIAGLNVLRIINEPTAAAIAYGLDKKVGAERNVLIFDLGGGTFDVSILTIE
DGIFEVKSTAGDTHLGGEDFDNRMVNHFIAEFKRKHKKDISENKRAVRRLRTAC
ERAKRTLSSSTQASIEIDSLYEGIDFYTSITRARFEELNADLFRGTLDPVEKALRDA
KLDKSQIHDIVLVGGSTRIPKIQKLLQDFFNGKELNKSINPDEAVAYGAAVQAAIL
SGDKSENVQDLLLLDVTPLSLGIETAGGVMTVLIKRNTTIPTKQTQTFTTYSDNQP
GVLIQVYEGERAMTKDNNLLGKFELTGIPPAPRGVPQIEVTFDIDANGILNVSAVD
KSTGKENKITITNDKGRLSKEDIERMVQEAEKYKAEDEKQRDKVSSKNSLESYAF
NMKATVEDEKLQGKINDEDKQKILDKCNEIINWLDKNQTAEKEEFEHQQKELEK
VCNPIITKLYQSAGGMPGGMPGGFPGGGAPPSGGASSGPTIEEVD
            The amino acid sequence of Hsp9A (SEQ ID NO: 21) is:
            MISASRAAARLPLLLPRGGPVPAVPGLAQTFWNGLSQNVLRAASSRKYAS
EAIKGAVIGIDLGTTNSCVAVMEGKQAKVLENSEGARTTPSVVAFTADGERLVG
MPAKRQAVTNPHNTFYATKRLIGRRFDDSEVKKDIKNVPFKIVRASNGDAWVEA
HGKLYSPSQIGAFVLMKMKETAENYLGHPAKNAVITVPAYFNDSQRQATKDAG
QISGLNVLRVINEPTAAALAYGLDKSEDKIIAVYDLGGGTFDISILEIQKGVFEVKS
TNGDTFLGGEDFDQALLQYIVKEFKRETSVDLTKDNMALQRVREASEKAKCELS
SSVQTDINLPYLTMDASGPKHLNMKLSRSQFEGIVADLIKRTVAPCQKAMQDAE
VSKSDIGEVILVGGMTRMPKVQQTVQDLFGRAPSKAVNPDEAVAIGAAIQGGVL
AGDVTDVLLLDVTPLSLGIETLGGVFTKLINRNTTIPTKKSQVFSTAADGQTQVEI
KVCQGEREMASDNKLLGQFTLVGIPPAPRGVPQIEVTFDIDANGIVHVSAKDKGT
GREQQIVIQSSGGLSKDEIENMVKNAEKYAEEDRRRKIERVEAVNLAEGIIHDTES
KMEEFKDQLPADECNKLKEEIAKMRELLARKDTETGENIRQAATSLQQASLKLFE
MAYKKMASE6RESSGSSGDQKEEK
```

Another embodiment of the present invention is the conjugate of a PTD with the low molecular weight heat shock or small stress protein cvHsp. In two-hybrid and co-immunoprecipitation experiments, cvHsp has been shown to bind the cytoskeleton protein α-filamin in the heart. The tissue distribution of α-filamin, characterized by highest expression in heart and skeletal muscle, is relevant to that of cvHsp. Within cvHsp, a domain of 64 amino acids (corresponding to amino acids 56-119) in the α-crystallin domain, was important for its interaction with filamin, suggesting that cvHsp acts as a chaperone protein. In addition, several genetic diseases with a pathophysiology compatible with the expression pattern and the putative role of cvHsp were mapped to chromosome 1p36.23-p34.3, a region associated with cardiomyopathy (see Krief et al., J Biol. Chem. 274:36592-36600 (1999)).

```
        The amino acid sequence of cvHsp
        (SEQ ID NO: 22) is:
        MSHRTSSTFRAERSFHSSSSSSSSSTSSSASRALPAQDPPMEKALS
MFSDDFGSFMRPHSEPLAFPARPGGAGNIKTLGDAYEFAVDVRDFSPEDI
IVTTSNNHIEVRAEKLAADGTVMNTFAHKCQLPEDVDPTSVTSALREDGS
LTIRARRHPHTEHVQQTFRTEIKI
```

The present invention also provides a conjugate of a PTD and a fragment, derivative or analogue of an Hsp polypeptide, such as a fragment, derivative or analogue of HspA1A, HspA1B, HspA1L, HspA2A, HspA2B, HspA4, HspA5, HspA6, HspA7, Hsp8A (Hsc70), Hsp9A, or cvHsp.

The peptide conjugates of the invention can be prepared by fusing a PTD-encoding gene with an Hsp gene and expressing the fusion protein in vitro or in vivo using standard cloning techniques and routine methods known to those having ordinary skill in the art.

The PTD-Hsp conjugate can be linked to each other by a direct covalent bond, a peptide bond, or a linker. Particularly, the PTD-Hsp conjugate can be linked to each other by a linker containing a region that is cleaved specifically by a certain enzyme. Linkers may vary depending on the purpose and the direction of therapy, and in order to maximize effects in local sites, a linker containing an —O— or —S—S— bond should be used, which is cleaved easily in cells. Linkers without a cleavage site (non-cleavage linkers) may also be used. The length of the linker is typically between 1 and 10 amino acids, preferably between 1 and 5 amino acids. The linker may contain the amino acids Gly-Gly-Gly. To avoid systemic effects, it is generally preferable to introduce a spacer linker containing a peptide bond. The linker can be amino caproic acid.

The use of PTD-Hsp mRNA for all of the above indications is also contemplated.

### DEFINITIONS

For convenience, certain terms used in the specification, examples, and appended claims are collected here. Unless otherwise defined, all technical and scientific terms used herein have the same meaning as commonly understood by one of ordinary skill in the art to which this invention pertains.

As used herein, by the term “ischemia” is meant an inadequate flow or shortage of blood to a part of the body, caused by constriction, obstruction or blockage of the blood vessels supplying it. Ischemia leads to tissue hypoxia. Hypoxia or ischemic-related injury includes cardiac injury.

As used herein, by the term “reperfusion” is meant the restoration of the flow of blood to a previously ischemic tissue or organ that has had its blood supply cut off, as after a heart attack or stroke.

As used herein, by the term “necrosis” is meant the death of cells or tissues through injury or disease, particularly in a localized area of the body such as the myocardium.

As used herein, by the term “apoptosis” is meant programmed cell death.

As used herein, the term “cardiac injury” is intended to encompass any chronic or acute pathological event involving the heart and/or associated tissues (e.g., the pericardium, aorta and other associated blood vessels), including, but not limited to, ischemia-reperfusion injury, congestive heart failure, cardiac arrest, myocardial infarction, cardiotoxicity caused by compounds such as drugs, cardiac damage due to parasitic infection, bacteria, fungi, rickettsiae, or viruses, fulminant cardiac amyloidosis, heart surgery, heart transplantation, and traumatic cardiac injury (e.g., penetrating or blunt cardiac injury, or aortic valve rupture).

As used herein, the term “neurodegenerative disease” is intended to encompass any degenerative event involving the brain, spinal column, nerves, and/or associated tissues, including, but not limited to, ischemia-reperfusion injury, neurotoxicity caused by compounds such as drugs, and neural damage due to parasitic infection.

As used herein, the term “vitrifying” means establishing a vitreous state in a solution and in cells, tissue or organs suspended in or perfused with that solution. A “vitreous state” is an amorphous solid formed from a liquid without the formation of crystals. As the term is used herein, a vitreous state refers more particularly to a solid formed from a liquid without the formation of ice crystals. Vitrification is accomplished by reducing the temperature of a solution below the glass transition temperature (Tg) for that solution when the Tg is lower than the homogeneous nucleation temperature for that solution, such that a vitreous state is established for the solution and for cells, tissue or organs suspended in or perfused with that solution. That is, “vitrification,” as it is used herein when a cell, tissue or organ is vitrified, occurs both inside cells, tissues or organs (i.e., inside the cells that comprise tissues and organs) and in the surrounding material (i.e., in the hypothermic storage solution). Vitreous storage is preferably performed at a temperature below the Tg for a hypothermic storage solution.

As used herein, the term “hypothermic storage solution” refers to a solution in which cells, tissues, or organs can be stored at temperatures below physiological temperature. Hypothermic storage solutions for the methods described herein have a Tg lower than the homogeneous nucleation temperature, such that the solution will form a glass, rather than a crystalline solid when temperature is reduced below the Tg. Vitrification, rather than crystal formation, occurs in a hypothermic storage solution due to the presence of one or more agents that inhibit ice crystal formation at temperatures higher than Tg. Hypothermic storage solutions having this property are known in the art. Preferred hypothermic storage solutions are described herein below. The term hypothermic storage solution does not include tissue culture growth medium alone.

As used herein, the term “inhibit” means to reduce an activity by at least 5%, and preferably more, e.g., 20%, 30%, 40%, 50%, 60%, 70%, 80%, 90% or more, up to and including 100% relative to that activity that is not subject to such inhibition. Thus, an agent that inhibits apoptosis by at least 5% relative to a sample subject to the same apoptotic stimulus but absent the agent.

As used herein, the term “polypeptide” is intended to encompass a singular “polypeptide” as well as plural “polypeptides,” and comprises any chain or chains of two or more amino acids joined together by peptide bonds. Thus, as used herein, terms including, but not limited to “peptide,” “dipeptide,” “tripeptide,” “protein,” “amino acid chain,” “oligopeptide,” “oligomer,” or any other term used to refer to a chain or chains of two or more amino acids, are included in the definition of a “polypeptide,” and the term “polypeptide” can be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids. The term “protein” is also intended to include fragments, analogues and derivatives of a protein wherein the fragment, analogue or derivative retains essentially the same biological activity or function as a reference protein.

The “fragment, derivative or analogue” of the protein may be (i) one in which one or more of the amino acid residues are substituted with a conserved or non-conserved amino acid residue (preferably, a conserved amino acid residue) and such substituted amino acid residue may or may not be one encoded by the genetic code; or (ii) one in which one or more of the amino acid residues includes a substituent group; or (iii) one in which the mature polypeptide is fused with another compound, such as a compound to increase the half life of the polypeptide (for example, polyethylene glycol); or (iv) one in which the additional amino acids are fused to the mature polypeptide, such as a leader or secretory sequence which is employed for purification of the polypeptide. Such fragments, derivatives and analogues are deemed to be within the scope of those skilled in the art from the teachings herein.

Particularly preferred are variants, analogues, derivatives and fragments having the amino acid sequence of the protein in which several, e.g., 5 to 10, 1 to 5, 1 to 3, 2, or 1 amino acid residues are substituted, deleted or added in any combination. Especially preferred among these are silent substitutions, additions and deletions, which do not alter the properties and activities of the protein of the present invention. Also especially preferred in this regard are conservative substitutions.

An example of a variant of the present invention is a fusion protein as defined above, apart from the substitution of one or more amino acids with one or more other amino acids. The skilled person is aware that various amino acids have similar properties. One or more such amino acids of a substance can often be substituted by one or more other such amino acids without eliminating a desired activity of that substance.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

The terms “fusion protein,” “fusion polypeptide,” “chimeric protein, and “chimeric polypeptide” as used herein are interchangeable and refer to polypeptides and proteins which comprise a polypeptide or protein of interest and a protein transduction domain (PTD).

The term PTD-Hsp “conjugate” as used herein refers to both the fusion of a PTD protein with an Hsp protein, as well as, the fusion of a PTD-encoding gene with an Hsp gene construct.

The terms “protein of interest”, “desired polypeptide”, “desired protein” or “target protein” as used herein are interchangeable and refer to a whole protein molecule or a portion thereof. The other portion of the polypeptide or protein is capable of inducing a cellular response.

As used herein, the term “therapeutic agent” refers to a molecule, such as a protein, lipid, carbohydrate, nucleic acid or chemical compound, which when delivered to a subject, treats, i.e., cures, ameliorates, or lessens the symptoms of, or inhibits a given disease or condition (e.g., ischemia or apoptosis) in that subject, or alternatively, prolongs the life of the subject by slowing the progress of a terminal disease or condition.

As used herein, the term “therapeutic fusion protein” refers to a polypeptide which when delivered to a subject, treats, i.e., cures, ameliorates, or lessens the symptoms of, a given disease or condition (e.g., ischemia or apoptosis) in that subject, or alternatively, prolongs the life of the subject by slowing the progress of a terminal disease or condition.

### Polypeptides

The therapeutic polypeptides of the present invention are the heat-shock proteins (Hsps). Hsps of the Hsp70 family are preferred. Examples of mammalian Hsps in the Hsp70 family include, but are not limited to, BIP (GRP78), mHSP70 (GRP75), HspA1A, HspA1B, HspA1L, HspA2A, HspA2B, HspA4, HspA5, HspA6, HspA7, Hsp8A (Hsc70), and Hsp9A. Hsps of the smHsp family are also preferred. Examples of smHsps family members include, but are not limited to, cvHsp, αB-crystallin, αA-crystallin, Hsp20, Hsp β-2, Hsp-like 27 and Hsp27.

Also included as polypeptides of the present invention are fragments, derivatives, analogs, or variants of the foregoing polypeptides, and any combination thereof, which are used to prevent or treat, i.e., cure, ameliorate, lessen the severity of, or reduce apoptotic conditions and/or neurodegenerative conditions or diseases.

Further embodiments of the invention include polypeptides, which comprise amino acid sequences at least 90% identical, and more preferably at least 91%, 92%, 93%, 94%, 95%, 96%, 97%, 98% or 99% identical, to any of the amino acid sequences of the polypeptides described above.

As a practical matter, whether any particular polypeptide is at least 90%, 91%, 92%, 93%, 94%, 95%, 96%, 97%, 98% or 99% identical to, for instance, the amino acid sequence shown in SEQ ID NO:11 can be determined conventionally using known computer programs such as the Bestfit program (Wisconsin Sequence Analysis Package, Version 8 for Unix, Genetics Computer Group, University Research Park 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acids in the reference sequence are allowed.

### Polynucleotides

Additionally, the present invention relates to polynucleotides which encode fusion proteins or chimeric proteins, recombinant expression vectors, plasmids and other polynucleotide constructs (collectively referred to as “expression vectors”) containing the same, microorganisms transformed with these expression vectors, and processes for obtaining these polynucleotides, and transformed cells using said vectors. Suitable host cells can be transformed with the expression vectors.

As used herein, the term “expression vector” refers to a construct made up of genetic material (i.e., nucleic acids). Typically, a expression vector contains an origin of replication which is functional in bacterial host cells, e.g., Escherichia coli, and selectable markers for detecting bacterial host cells comprising the expression vector. Expression vectors of the present invention contain a promoter sequence and include genetic elements as described herein arranged such that an inserted coding sequence can be transcribed and translated in eukaryotic cells. In certain embodiments described herein, an expression vector is a closed circular DNA molecule.

The term “expression” refers to the biological production of a product encoded by a coding sequence. In most cases, a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

The fusion proteins or chimeric proteins of this invention can be prepared by recombinant DNA methodology. In accordance with the present invention, a gene sequence coding for a desired protein is isolated, synthesized or otherwise obtained and operably linked to a DNA sequence coding for the PTD peptide. The hybrid gene containing the gene for a desired protein operably linked to a DNA sequence encoding a PTD peptide is referred to as a chimeric gene. Optionally, the gene sequence coding for a desired protein may be operably linked to the DNA sequence coding for the PTD peptide via a linker sequence.

The term “linker peptide” is intended to define any sequence of amino acid residues which preferably provide a hydrophilic region when contained in an expressed protein. Such a hydrophilic region may facilitate cleavage by an enzyme at the proteolytic cleavage site.

The chimeric gene is inserted into an expression vector which allows for the expression of the desired chimeric protein in a suitable transformed host. The expression vector provides the inserted chimeric gene with the necessary regulatory sequences to control expression in the suitable transformed host.

The nucleic acid construct may be in the form of a vector, for example, an expression vector, and may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host, may be used for expression in this regard.

Regulatory elements that control expression of the fusion protein of the present invention include the promoter region, the 5′ untranslated region, the signal sequence, the chimeric coding sequence, the 3′ untranslated region, and the transcription termination site. Fusion proteins which are to be secreted from a host into the medium also contain the signal sequence.

Similarly, a variety of translation control elements are known to those of ordinary skill in the art. These include, but are not limited to ribosome binding sites, and translation initiation and termination codons.

Methods and materials for preparing recombinant vectors and transforming host cells using the same, replicating the vectors in host cells and expressing biologically active foreign polypeptides and proteins are described in Principles of Gene Manipulation, by Old and Primrose, 2nd edition (1981), and Sambrook et al., Molecular Cloning, 3rd edition, Cold Spring Harbor Laboratory (2001), both incorporated herein by reference.

As used herein, the term “DNA polynucleotide” may be a circular or linearized plasmid, or other linear DNA which may also be non-infectious and nonintegrating (i.e., does not integrate into the genome of vertebrate cells). A linearized plasmid is a plasmid that was previously circular but has been linearized, for example, by digestion with a restriction endonuclease. Linear DNA may be advantageous in certain situations as discussed, e.g., in Chemg, J. Y., et al., J Control. Release 60:343-353 (1999), and Chen, Z. Y., et al., Mol. Ther. 3:403-410 (2001), both of which are incorporated herein by reference.

Further embodiments of the invention include vectors comprising chimeric genes, which comprise a nucleotide at least 90% identical, and more preferably at least 91%, 92%, 93%, 94%, 95%, 96%, 97%, 98% or 99% identical, to any of the nucleotide sequences of the vectors comprising chimeric genes described above.

Other embodiments of the invention include chimeric genes, which comprise a nucleotide sequence at least 90% identical, and more preferably at least 91%, 92%, 93%, 94%, 95%, 96%, 97%, 98% or 99% identical, to any of the nucleotide sequences of the chimeric genes described above.

As a practical matter, whether any particular vector or chimeric gene is at least 91%, 92%, 93%, 94%, 95%, 96%, 97%, 98% or 99% identical to a nucleotide sequence according to the present invention, can be determined conventionally using known computer programs such as the Bestfit program (Wisconsin Sequence Analysis Package, Version 8 for Unix, Genetics Computer Group, University Research Park 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

### Codon Optimization

“Codon optimization” is defined as modifying a nucleic acid sequence for enhanced expression in the cells of the subject of interest, e.g., human, by replacing at least one, more than one, or a significant number, of codons of the native sequence with codons that are more frequently or most frequently used in the genes of that subject. Various species exhibit particular bias for certain codons of a particular amino acid.

In one aspect, the present invention relates to polynucleotide expression constructs or vectors, and host cells comprising nucleic acid fragments of codon-optimized coding regions which encode therapeutic polypeptides, and fragments, variants, or derivatives thereof, and various methods of using the polynucleotide expression constructs, vectors, host cells to treat or prevent disease in a subject.

As used herein the term “codon-optimized coding region” means a nucleic acid coding region that has been adapted for expression in the cells of a given subject by replacing at least one, or more than one, or a significant number, of codons with one or more codons that are more frequently used in the genes of that subject.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). Many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

### Consensus Sequences

The present invention is further directed to expression plasmids that contain chimeric genes which express therapeutic fusion proteins with specific consensus sequences, and fragments, derivatives and variants thereof. A “consensus sequence” is, e.g., an idealized sequence that represents the amino acids most often present at each position of two or more sequences which have been compared to each other. A consensus sequence is a theoretical representative amino acid sequence in which each amino acid is the one which occurs most frequently at that site in the different sequences which occur in nature. The term also refers to an actual sequence which approximates the theoretical consensus. A consensus sequence can be derived from sequences which have, e.g., shared functional or structural purposes. It can be defined by aligning as many known examples of a particular structural or functional domain as possible to maximize the homology. A sequence is generally accepted as a consensus when each particular amino acid is reasonably predominant at its position, and most of the sequences which form the basis of the comparison are related to the consensus by rather few substitutions, e.g., from 0 to about 100 substitutions. In general, the wild-type comparison sequences are at least about 50%, 75%, 80%, 90%, 95%, 96%, 97%, 98% or 99% identical to the consensus sequence. Accordingly, polypeptides of the invention are about 50%, 75%, 80%, 85%, 90%, 91%, 92%, 93%, 94%, 95%, 96%, 97%, 98%, 99% or 100% identical to the consensus sequence.

A “consensus amino acid” is an amino acid chosen to occupy a given position in the consensus protein. A system which is organized to select consensus amino acids can be a computer program, or a combination of one or more computer programs with “by hand” analysis and calculation. When a consensus amino acid is obtained for each position of the aligned amino acid sequences, then these consensus amino acids are “lined up” to obtain the amino acid sequence of the consensus protein.

### Therapeutic Uses

Apoptotic and necrotic cell death, and other programmed cell death pathways are often involved in ischemic brain injury, heart disease, and neurodegenerative disease. The therapeutic fusion proteins described above may be used in the manufacture of a medicament to effectively suppress apoptosis and the development of diseases caused by apoptosis.

The therapeutic fusion proteins of the invention may also be co-administered with one or more compounds or constructs. Other compounds include, but are not limited to, anti-platelet drugs, anti-coagulant drugs, or anti-thrombotic drugs, caspase-inhibitors, as well as other polypeptides, including members of the Hsp family (e.g., co-chaperone Hsp40).

The therapeutic fusion proteins of the invention may be targeted to the following cells or cell types: stem cells (e.g., hematopoietic, mesenchymal, stromal or neural stem cells), cardiovascular cells, such as cardiac myocytes, ventricular myocytes, atrial myocytes, cardiac stem cells, endothelial cells, vascular smooth muscle cells, pacemaker cells, myofibroblasts or fibroblasts, neural cells, such as neurons (also called nerve cells or neurocytes), tumor cells, macrophages, epithelial cells, keratinocytes, granulocytes, erythrocytes, lymphocytes or platelets. The cells may be differentiated or precursor cells.

A series of specific treatments applicable to mesenchymal stem cells (MSCs) to induce expression of cardiac specific genes are disclosed herein. The conditions are effective on rat, canine and human MSCs. Treatments of MSCs include (1) co-culturing MSCs with fetal, neonatal and adult rat cardiac cells; (2) use of chemical fusigens (e.g., polyethylene glycol or sendai virus) to create heterokaryons of MSCs with fetal, neonatal and adult cardiomyocytes; (3) incubating MSCs with extracts of mammalian hearts, including the extracellular matrix and related molecules found in heart tissue; (4) treatment of MSCs with growth factors and differentiating agents; (5) mechanical and/or electrical stimulation of MSCs, and (6) mechanically and/or electrically coupling MSCs with cardiomyocytes. MSCs that progress towards cardiomyocytes first express proteins found in fetal cardiac tissue and then proceed to adult forms. Detection of expression of cardiomyocyte specific proteins is achieved using antibodies to, for example, myosin heavy chain monoclonal antibody MF 20, sarcoplasmic reticulum calcium ATPase (SERCA1) (mnAb 10D1) or gap junctions using antibodies to connexin 43.

Cardiac injury promotes tissue responses which enhance myogenesis using implanted MSCs. Thus, MSCs are introduced to the infarct zone to reduce the degree of scar formation and to augment ventricular function. New muscle is thereby created within an infarcted myocardial segment. MSCs are directly infiltrated into the zone of infarcted tissue. The integration and subsequent differentiation of these cells is characterized and timing of intervention is designed to mimic the clinical setting where patients with acute myocardial infarction would first come to medical attention, receive first-line therapy, followed by stabilization, and then intervention with myocardial replacement therapy if necessary.

Of the four chambers of the heart, the left ventricle is primarily responsible for pumping blood under pressure through the body's circulatory system. It has the thickest myocardial walls and is the most frequent site of myocardial injury resulting from congestive heart failure. The degree of advance or severity of the congestive heart failure ranges from those cases where heart transplantation is indicated as soon as a suitable donor organ becomes available to those where little or no permanent injury is observed and treatment is primarily prophylactic.

The severity of resulting myocardial infarction, i.e., the percentage of muscle mass of the left ventricle that is involved can range from about 5 to about 40 percent. This represents affected tissue areas, whether as one contiguous ischemia or the sum of smaller ischemic lesions, having horizontal affected areas from about 2 cm² to about 6 cm² and a thickness of from 1-2 mm to 1-1.5 cm. The severity of the infarction is significantly affected by which vessel(s) is involved and how much time has passed before treatment intervention is begun.

The mesenchymal stem cells used in accordance with the invention are, in order of preference, autologous, allogeneic or xenogeneic, and the choice can largely depend on the urgency of the need for treatment. A patient presenting an imminently life threatening condition may be maintained on a heart/lung machine while sufficient numbers of autologous MSCs are cultured or initial treatment can be provided using other than autologous MSCs.

Methods and Administration

The present invention provides methods for delivery of a therapeutic fusion protein, or a fragment, variant, or derivative thereof, in admixture with one or more pharmaceutically acceptable carriers or excipients. The therapeutic fusion protein is provided as a recombinant protein, in particular, a fusion protein, or a purified subunit, which comprises administering to a subject one or more of the compositions described herein; such that upon administration of compositions such as those described herein, a therapeutic response is generated in a subject. The delivery can occur, for example, through the skin, nose, eye, into muscle, brain or heart, or by intravenous injection.

The term “subject” is intended to encompass living organisms such as humans, monkeys, cows, sheep, horses, pigs, cattle, goats, dogs, cats, mice, rats, cultured cells therefrom, and transgenic species thereof. In a preferred embodiment, the subject is a human.

The term “vertebrate” is intended to encompass a singular “vertebrate” as well as plural “vertebrates” and comprises mammals and birds, as well as fish, reptiles, and amphibians.

The term “mammal” is intended to encompass a singular “mammal” and plural “mammals,” and includes, but is not limited to humans; primates such as apes, monkeys (e.g., owl, squirrel, cebus, rhesus, African green, patas, cynomolgus, and cercopithecus), orangutans, baboons, gibbons, and chimpanzees; canids such as dogs and wolves; felids such as cats, lions, and tigers; equines such as horses, donkeys, and zebras, food animals such as cows, pigs, and sheep; ungulates such as deer and giraffes; ursids such as bears; and others such as rabbits, mice, ferrets, seals, whales. In particular, the mammal can be a human subject, a food animal or a companion animal.

The term “bird” is intended to encompass a singular “bird” and plural “birds,” and includes, but is not limited to feral water birds such as ducks, geese, terns, shearwaters, and gulls; as well as domestic avian species such as turkeys, chickens, quail, pheasants, geese, and ducks. The term “bird” also encompasses passerine birds such as starlings and budgerigars.

The present invention further provides a method for generating, enhancing or modulating a therapeutic response comprising administering to a human one or more of the compositions described herein. In this method, the compositions may include one or more polypeptides, or a fragment, variant, or derivative thereof, wherein the protein is provided as a recombinant protein, in particular, a fusion protein, or a purified subunit.

As used herein, a “therapeutic response” refers to the ability of a subject to elicit a positive reaction to a composition, as disclosed herein, when delivered to that subject.

As mentioned above, compositions of the present invention can be used to therapeutically treat and prevent disease or disease conditions. As defined herein, “treatment” refers to the use of one or more compositions of the present invention to prevent, cure, retard, or reduce the severity of a disease or disease symptoms in a subject, and/or result in no worsening of the disease.

The diseases or disease conditions caused by or leading to apoptosis that are contemplated as part of this invention include, but are not limited to, ischemia/hypoxia, such as cardiac hypoxia, cardiac hypoxia-reoxygenation, cardiac ischemia-reperfusion injury, ischemic heart disease, heart failure, heart hypertrophy, heart surgery, traumatic heart injury, coronary angioplasty, vascular defects or blockages (obstruction of blood flow), congenital heart disease, congestive heart failure, cardiac cell muscle regeneration, chemotherapeutic induced cardiomyophathy, myocardial infarction, cardiac arrest, cardiotoxicity, cardiac damage due to parasitic infection, fulminant cardiac amyloidosis, cardiac transplantation, or traumatic cardiac or brain injury, stroke due to ischemic cerebral infarction, ischemic or hemorrhagic stroke, ischemic acute renal failure, intestinal ischemia, ischemic heart disease due to myocardial infarction (myocardial ischemia and disorder after reperfusion, liver ischemia, brain ischemia (e.g., brain ischemia from apoplexy and the like), frost damage and ischemia retinae, intracranial bleedings (subarachnoid hemorrhage, thrombolytica-induced etc.), blood clots, hypoxia-induced apoptosis, and tissue damage following ischemia-reperfusion.

Additional degenerative diseases of the heart include, but are not limited to, viral myocarditis, autoimmune myocarditis (congestive cardiomyopathy and chronic myocarditis), myocardial disorders or death due to hypertrophic heart and heart failure, arrythmogenic right ventricular cardiomyopathy, heart failure, and coronary artery by-pass graft.

Ischemia of the neuroretina and optic nerve can arise during retinal branch vein occlusion, retinal branch artery occlusion, central retinal artery occlusion, central retinal vein occlusion, during intravitreal surgery, in retinal degenerations such as retinitis pigmentosa, and age-related macular degeneration.

Neurodegenerative diseases or disease conditions caused by or leading to apoptosis that are contemplated as part of this invention include, but are not limited to, myasthenia gravis, Alzheimer's disease, Parkinsonian Syndromes, including Parkinson's disease, Huntington's disease, multiple sclerosis, amyotrophic lateral sclerosis or motor neuron disease (ALS), spinobulbar atrophy, denervation atrophy, spinal muscular dystrophy (SMA), pigmentary degeneration of the retina and glaucoma, cerebellar degeneration and neonatal jaundice, otosclerosis, stroke, dementia, successive delayed neuronal death (DND). Motor Neuron Disease (ALS), diffuse cerebral cortical atrophy, Lewy-body dementia, Pick disease, mesolimbocortical dementia, thalamic degeneration, bulbar palsy, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, diabetic retinopathy, and opthalmoplegia. The skilled person understands that these and other mild, moderate or severe neurodegenerative conditions can be treated according to a method of the invention.

Other degenerative diseases caused by or leading to apoptosis include, but are not limited to, degenerative atrophy, alcoholic hepatitis, viral hepatitis, renal diseases (e.g., glomerulonephritis), hemolytic uremic symdrome and the like, acquired immunodeficiency syndrome (AIDS), inflammatory skin disorders such as toxic epidermal necrolysis (TEN) and multiform exudative erythema, graft versus host disease (GVH), radiation disorders, side effects due to anti-cancer drugs, anti-viral drugs and the like, disorders due to toxic agents such as sodium azide, potassium cyanide and the like, osteomyelo-dysplasia such as aplastic anemia and the like, prion diseases such as Creutzfeldt-Jakob's disease, spinal cord injury, traumatic brain injury, cytotoxic T cell or natural killer cell-mediated apoptosis associated with autoimmune disease and transplant rejection, mitochondrial drug toxicity, e.g., as a result of chemotherapy or HIV therapy, viral, bacterial, or protozoal infection, inflammation or inflammatory diseases, inflammatory bowel disease, sepsis and septic shock, follicule to ovocyte stages, from ovocyte to mature egg stages and sperm (e.g., methods of freezing and transplanting ovarian tissue, artificial fecondation), skin damage (due to exposure to high level of radiation, heat, burns, chemicals, sun, and autoimmune diseases), myelodysplastic syndromes (MDS) (death of bone marrow cells), pancreatitis, osteoarthritis, rheumatoid arthritis, psoriasis, glomerulonephritis, atherosclerosis, and graft versus host disease, retinal pericyte apopotosis, retinal neurons apoptosis glaucoma, retinal damages resulting from ischemia, diabetic retinopathy, respiratory syndrome, diabetes (e.g., insulin dependent diabetes), autoimmune disease, acquired poly glutamine disease, Monckeberg's, encephalopathy associated with acquired immunodeficiency disease (AIDS), myopathies and muscular dystrophies, glomerulosclerosis, Monckeberg's medial sclerosis, inflammatory bowel disease, Crohn's disease, autoimmune hepatitis, hemochromatosis and Wilson disease, alcoholic hepatitis, acute hepatic failure of different etiology, diseases of the bile ducts, atherosclerosis, hypertension, apoptosis-induced hair loss and apoptosis associated with the use of chemotherapeutic drugs.

The term “prevention” refers to the use of one or more compositions of the present invention to generate a therapeutic responses in a subject. It is not required that any composition of the present invention totally cure or eliminate all disease symptoms.

In certain embodiments, one or more compositions of the present invention are delivered to a subject by methods described herein, thereby achieving an effective therapeutic response. More specifically, the compositions of the present invention may be administered to any tissue of a subject, including, but not limited to, skin, muscle, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, and connective tissue, e.g., cartilage. The preferred tissues are heart and brain tissue.

The mesenchymal stem cell (MSC) therapy of the invention can be provided by several routes of administration, including the following. First, intracardiac muscle injection, which avoids the need for an open surgical procedure, can be used where the MSCs are in an injectable liquid suspension preparation or where they are in a biocompatible medium which is injectable in liquid form and becomes semi-solid at the site of damaged myocardium. A conventional intracardiac syringe or a controllable arthroscopic delivery device can be used so long as the needle lumen or bore is of sufficient diameter (e.g., 30 gauge or larger) that shear forces will not damage the MSCs. The injectable liquid suspension MSC preparations can also be administered intravenously, either by continuous drip or as a bolus. During open surgical procedures, involving direct physical access to the heart, all of the described forms of MSC delivery preparations are available options.

As a representative example of a dose range is a volume of about 20 to about 50 ul of injectable suspension containing 10-40×10⁶ MSCs/ml. The concentration of cells per unit volume, whether the carrier medium is liquid or solid remains within substantially the same range. The amount of MSCs delivered will usually be greater when a solid, “patch” type application is made during an open procedure, but follow-up therapy by injection will be as described above. The frequency and duration of therapy will, however, vary depending on the degree (percentage) of tissue involvement (e.g., 5-40% left ventricular mass).

In cases having in the 5-10% range of tissue involvement, it is possible to treat with as little as a single administration of one million MSCs in 20-50 μl of injection preparation. The injection medium can be any pharmaceutically acceptable isotonic liquid. Examples include phosphate buffered saline (PBS), culture media such as DMEM (preferably serum-free), physiological saline or 5% dextrose in water.

In cases having more in a range around the 20% tissue involvement severity level, multiple injections of 20-50 μl (10-40×10⁶ MSCs/ml) are envisioned. Follow-up therapy may involve additional dosings.

In very severe cases, e.g., in a range around the 40% tissue involvement severity level, multiple equivalent doses for a more extended duration with long term (up to several months) maintenance dose aftercare may well be indicated.

Furthermore, the compositions of the present invention may be administered to any internal cavity of a subject, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland or a liver. When the compositions of the present invention is administered to the lumen of a duct of a salivary gland or liver, the desired polypeptide is expressed in the salivary gland and the liver such that the polypeptide is delivered into the blood stream of the subject from each of the salivary gland or the liver. Certain modes for administration to secretory organs of a gastrointestinal system using the salivary gland, liver and pancreas to release a desired polypeptide into the bloodstream is disclosed in U.S. Pat. Nos. 5,837,693 and 6,004,944, both of which are incorporated herein by reference in their entireties.

According to the disclosed methods, compositions of the present invention can be administered by injection, intravenous, intramuscular (i.m.), subcutaneous (s.c.), or intrapulmonary routes. Other suitable routes of administration include, but are not limited to intratracheal instillation, transdermal, intraocular, intranasal, inhalation, intracavity, intraductal (e.g., into the pancreas) and intraparenchymal (i.e., into any tissue) administration. For intravenous administration, appropriate pharmaceutically acceptable carriers can be used, such as phosphate buffered saline, saline, or other materials used for administration of drugs intravenously. Transdermal delivery includes, but is not limited to intradermal (e.g., into the dermis or epidermis), transdermal (e.g., percutaneous) and transmucosal administration (i.e., into or through skin or mucosal tissue). Intracavity administration includes, but is not limited to administration into oral, vaginal, rectal, nasal, peritoneal, or intestinal cavities as well as, intrathecal (i.e., into the spinal canal), intraventricular (i.e., into the brain ventricles or the heart ventricles), intra-atrial (i.e., into the heart atrium) and sub arachnoid (i.e., into the sub arachnoid spaces of the brain) administration.

Any mode of administration can be used so long as the mode results in delivery or the expression of the desired peptide or protein, in the desired tissue, in an amount sufficient to generate a therapeutic response to a disease condition in a human in need of such a response.

Administration means of the present invention include needle injection (for example as a sterile aqueous dispersion, preferably isotonic), transdermal, catheter infusion, biolistic injectors, particle accelerators (e.g., “gene guns” or pneumatic “needleless” injectors) Med-E-Jet (Vahlsing, H., et al., J. Immunol. Methods 171:11-22 (1994)), Pigjet (Schrijver, R., et al., Vaccine 15:1908-1916 (1997)), Biojector (Davis, H., et al., Vaccine 12:1503-1509 (1994); Gramzinski, R., et al., Mol. Med. 4:109-118 (1998)), AdvantaJet (Linmayer, I., et al., Diabetes Care 9:294-297 (1986)), Medi-jector (Martins, J., and Roedl, E. J., Occup. Med. 21:821-824 (1979)), gelfoam sponge depots, other commercially available depot materials (e.g., hydrogels), osmotic pumps (e.g., Alza minipumps), oral or suppositorial solid pharmaceutical formulations, such as tablets, pills, soft and hard capsules, liquids, suspensions, syrups, granules and elixers, topical skin creams or gels, and decanting, use of polynucleotide coated suture (Qin, Y., et al., Life Sciences 65:2193-2203 (1999)) or topical applications during surgery.

Certain modes of administration are intramuscular needle-based injection and pulmonary application via catheter infusion. Energy-assisted plasmid delivery (EAPD) methods may also be employed to administer the compositions of the invention. One such method involves the application of brief electrical pulses to injected tissues, a procedure commonly known as electroporation. See generally Mir, L. M., et al., Proc. Natl. Acad. Sci USA 96:4262-7 (1999); Hartikka, J., et al., Mol. Ther. 4:407-15 (2001); Mathiesen, I., Gene Ther. 6:508-14 (1999); Rizzuto G., et al., Hum. Gen. Ther. 11:1891-900 (2000). Each of the references cited in this paragraph is incorporated herein by reference in its entirety.

Determining an effective amount of one or more compositions of the present invention depends upon a number of factors including, for example, the fusion protein, variants, or derivatives thereof being expressed or administered directly, the age, weight and sex of the subject, the precise condition requiring treatment and its severity, the route of administration, the in vivo half-life of the fusion protein, the efficiency of uptake, and the area to be treated. Treatment can be repeated as necessary, based on clinical judgment, in view of patient response.

A “pharmaceutically effective amount” or a “therapeutically effective amount” is an amount sufficient to generate a therapeutic or clinical response to a disease condition. The terms “pharmaceutically effective amount” or a “therapeutically effective amount are interchangeable. Based on the above factors, determining the precise amount, number of doses, and timing of doses are within the ordinary skill in the art and will be readily determined by the attending physician or veterinarian.

For administration to mammals, and particularly humans, it is expected that the daily dosage of the active agent will be from 0.01 mg/kg body weight, typically around 1 mg/kg. The above dosages are exemplary of the average case. There can, of course, be instances where higher or lower dosages are merited, including picomolar and nanomolar concentrations, and such are within the scope of this invention.

The present invention also relates to compositions comprising the fusion protein(s), as disclosed herein, and an additional pharmaceutically active agent. The fusion protein(s) and associated pharmaceutically active agent may be employed in combination with pharmaceutically acceptable one or more carriers or excipients. Such carriers may include, but are not limited to, diluents (e.g., lactose, dextrose, sucrose, mannitol, sorbitol, cellulose and/or glycine), lubricants (e.g., silica, talc, stearic acid and polyethylene glycol), binders (e.g., magnesium aluminum silicate, starch paste, gelatin, tragacanth, methylcellulose, sodium carboxymethylcellulose and/or polyvinylpyrrolidone), and disintegrants, such as starches, agar, alginic acid, or its sodium salt, and/or absorbents, colorants, flavors, and sweeteners, saline, buffered saline, liposomes, water, glycerol, ethanol and combinations thereof.

Compositions of the present invention may be solubilized in any of various buffers. Suitable buffers include, for example, phosphate buffered saline (PBS), normal saline, Tris buffer, and sodium phosphate (e.g., 150 mM sodium phosphate). Insoluble polynucleotides may be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a human.

Compositions of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995), both of which are incorporated herein by reference in their entireties. Although the composition may be administered as an aqueous solution, it can also be formulated as an emulsion, gel, solution, suspension, lyophilized form, or any other form known in the art.

The following examples are included for purposes of illustration only and are not intended to limit the scope of the present invention, which is defined by the appended claims. All references cited in the Examples are incorporated herein by reference in their entireties.

### EXAMPLES

### Example 1

### Preparation of Expression Vector Containing PTD-HspA1A

In order to link a base sequence encoding HSPA1A with a base sequence encoding a peptide region from the 858th amino acid (tyrosine) to the 868th amino acid (arginine) from the N-terminus of human transcription factor Hph-1 (GenBank Accession No: U63386), the primers having the following base sequences were synthesized: a base sequence corresponding to restriction enzyme EcoRI for cloning into a pET28B(+) vector having a base sequence from the 858th amino acid (tyrosine) to 868th amino acid (arginine) from the N-terminus of Hph-1; and a base sequence corresponding to restriction enzyme HindIII for cloning with sequences corresponding to the 5′-terminus and 3′-terminus of the base sequence of HSPA1A. PCR was performed using the above primers, a pRS vector (commercially available from Invitrogen) containing the whole gene of the HSPA1A protein, as a template, and pfu turbo DNA polymerase (Stratagene, cat.# 600252-51).

The PCR reaction product was cut with restriction enzymes EcoRI and HindIII, and purified with the Quiaquick PCR purification kit (QIAGEN, cat.# 28104). The purified product was cloned into the BglII site of pET28B(+) (commercially available from Invitrogen, Cat. No. V360-20B). The prepared recombinant vector was named “pHph-2-Hsp70”.

### Example 2

### Preparation of E. coli Transform Ants and Expression and Purification of Fusion Protein

E. coli BL21-DE3 (ATCC No. 53863) was transformed with the expression vector pHph-2-HSP70 prepared in Example 1, by heat shock transformation, and the transformed E. coli strain was inoculated into 4 ml of LB medium and pre-cultured at 37° C. for 14 hours with stirring. Then, the pre-culture medium was inoculated into 250 ml of LB medium (10 g/l casein pancreatic digest, 5 g/l yeast extract, 10 g/l sodium chloride), and cultured at 37° C. for 3 hours. Then, 1 mM IPTG (isopropyl β-D-thiogalactopyranoside; GibcoBRL cat.# 15529-019) was added to the culture medium, and the mixture was cultured at 37° C. for 4 hours to induce the expression of a fusion protein. The culture medium was centrifuged at 4° C. and 6,000 rpm for 20 minutes, and the supernatant was removed, leaving pellets. The pellets were dissolved in 10 ml of buffer solution 1 (50 mM NaH2PO4, 300 mM NaCl, 10 mM imidazole, pH 8.0) and sonicated with an ultrasonic processor (Heat systems, ultrasonic processor XL) on ice at an intensity of 300 W for 6 seconds and then cooled. The sonication and cooling steps were repeated such that the total sonication time reached 8 minutes. The lysate was centrifuged at 4° C. and 12,000 rpm for 10 minutes, and the disrupted E. coli cells were removed and only a pure lysate was collected. To the collected lysate, 0.5 ml of 50% Ni2+-NTA agarose slurry (Qiagen, cat# 30230) was added, and the suspension was stirred at 4° C. at 200 rpm for 1 hour, such that the fusion protein and the Ni2+-NTA agarose were bound to each other. The mixture was passed through a 0.8×4 cm chromatography column (BioRad, cat.# 731-1550). The resulting material was washed two times with 4 ml of buffer solution 2 (20 mM Tris-HCl, 500 mM NaCl, 20 mM imidazole, pH 7.9), and treated with 1 ml of buffer solution 3 (50 mM NaH2PO4, 300 mM NaCl, 300 mM imidazole, pH 8.0), thus obtaining a fusion protein fraction. The fraction was desalted with a PD-10 desalting column (Amersham-Pharmacia Biotech cat.# 17-0851-01). The isolated and purified PTD-HSPA1A fusion protein was subjected to SDS-PAGE, and then analyzed by Coomassie blue staining.

### Example 3

### Apoptosis Suppressing Effect of PTD-HspA1A

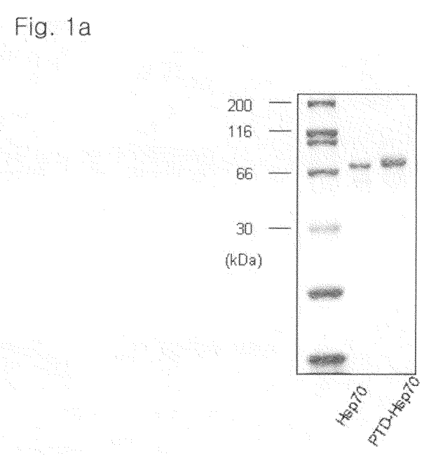

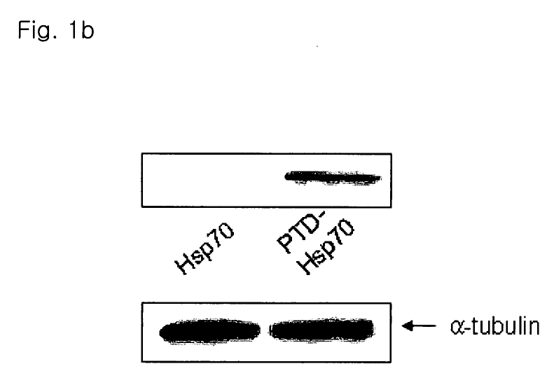

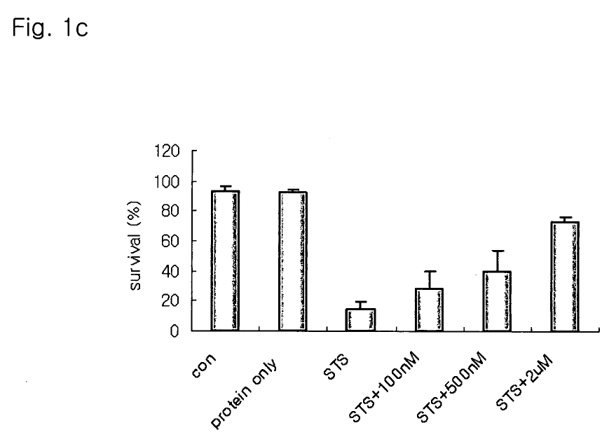

An HspA1A protein and a PTD-conjugated HspA1A protein were purified (FIG. 1A), 1 μl of each of the proteins was added to a medium with Jurkat T cells and cultured for 1 hour. As a result, it could be observed that only the PTD-conjugated protein was introduced into the cells (FIG. 1B). Also, cells were treated with 0.5 μM staurosporin (STS) to induce apoptosis, various concentrations of the PTD-HspA1A were added, and the cells analyzed for the degree of apoptosis. The results showed that the PTD-Hsp70 exhibited an apoptosis-suppressing effect in a concentration-dependent manner (FIG. 1C). In FIG. 1C, con represents Jurkat T cell only, and STS represents staurosporin.

### Example 4

### Apoptosis-Suppressing Effect of PTD-HspA1A Under Low-Oxygen Conditions

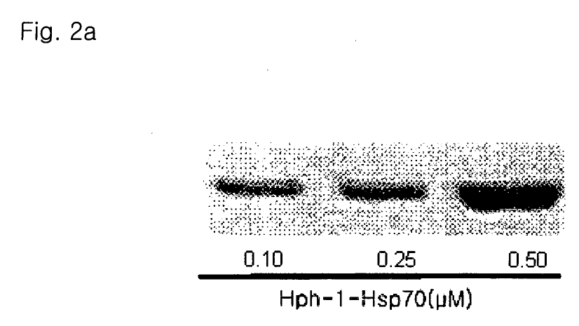

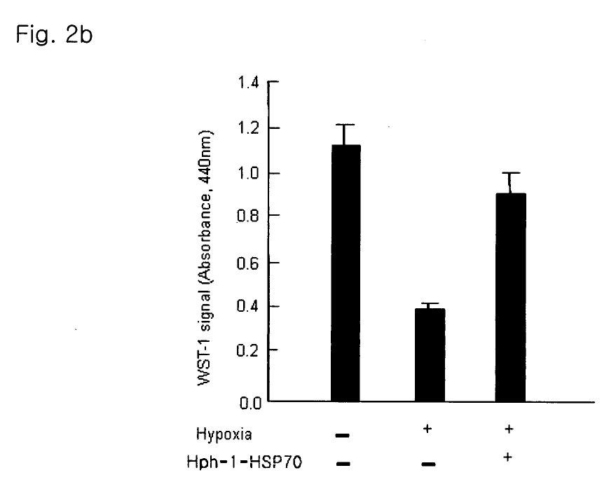

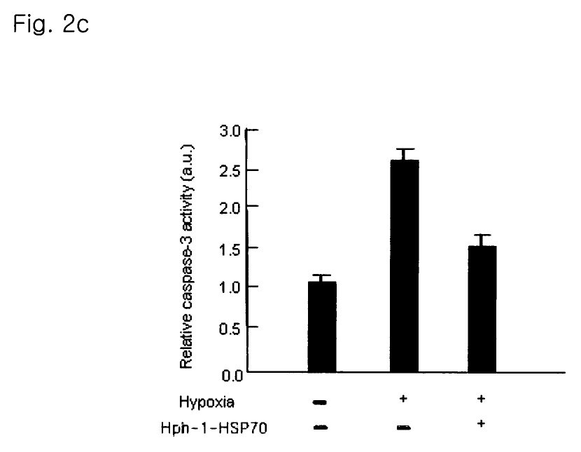

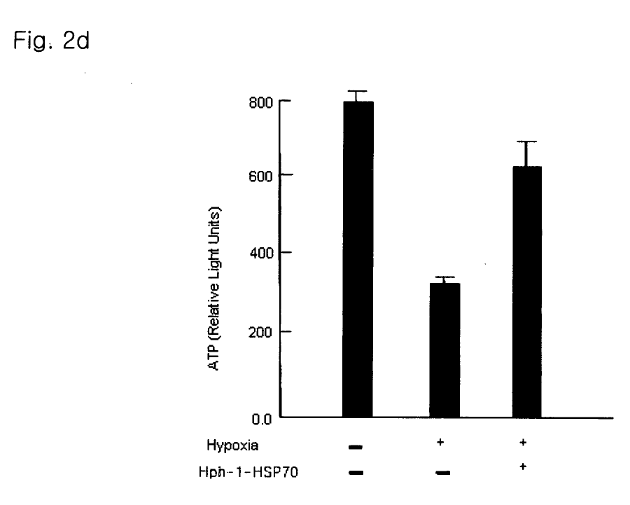

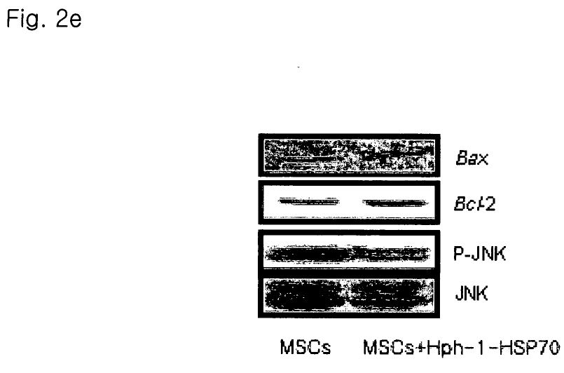

The PTD-HspA1A was introduced into mesenchymal stem cells (MSC) under low-oxygen conditions and examined for its apoptosis-suppressing effect. FIG. 2A shows the introduction of various concentrations of the purified PTD-HspA1A into MSC. It was observed that the apoptosis of MSC under low-oxygen conditions was suppressed in the presence of HspA1A (FIGS. 2B, 2C and 2D). In particular, the apoptosis of MSC under low-oxygen conditions (hypoxia) was suppressed in the presence of HspA1A as shown by an increased WST-1 signal. Tetrazolium salts (WST-1) are cleaved to formazan by the succinate-tetrazolium reductase system, which belongs to the respiratory chain of the mitochondria. As cell population increases, an increase in the amount of reductase present in the culture supernatant results in a concomitant increase in the conversion of WST-1 to formazan dye. It was also shown that the introduction of HspA1A suppressed the expression of a Bax protein, and inhibited the phosphorylation (i.e., activation) of a JNK (c-Jun N-terminal kinase, stress activated protein kinase) protein while maintaining the expression level thereof, thus suppressing apoptosis (FIG. 2E).

### Example 5

### Apoptosis-Suppressing Effect of PTD-HspA1A in Retinal Degeneration Model

Sprague-Dawley rats (n=3 per group) received an intraperitoneal injection of 60 mg/kg MNU (N-methyl-N-nitrosourea), which was immediately followed by intraperitoneal injection of 1 mg PTD-hspA1A. The injection was repeated at 24-hour intervals (just after electroretinogram up to 72 hours) for 6 days.

At 24 hours, 48 hours, 72 hours and 6 days after the start of the experiment, ERG (electroretinogram) was carried out. At 7 days, deep anesthesia was induced and the chest cavity of the rat was opened and the rat was fixed by transcardiac perfusion with 4% PFA (paraformaldehyde), and then the eyeball was isolated and immersed in 4% PFA. Then, the anterior eye segment was excised and the posterior eye cup was embedded in a paraffin block and sectioned to a thickness of 6 μm, and the sections were stained with H&E (hematoxilin and eosin) and observed for the change in the retinal layer.

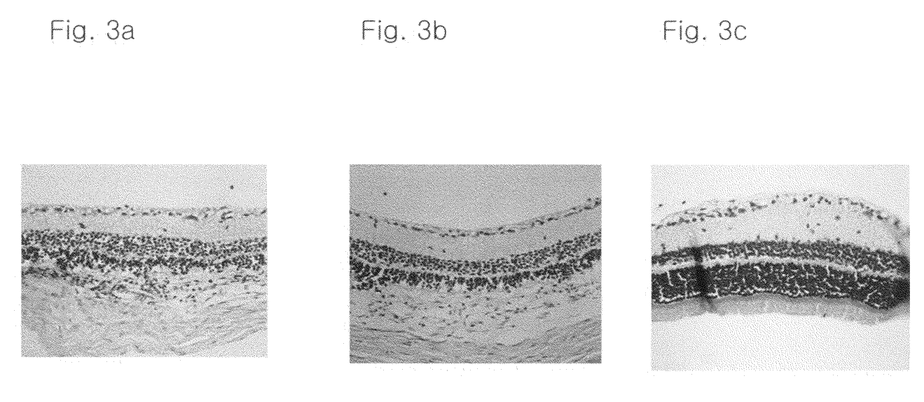

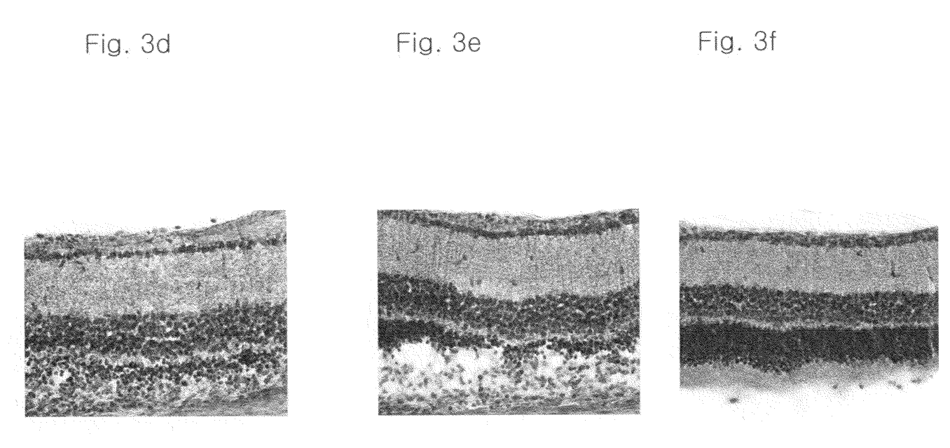

In a retinal degeneration model having apoptosis induced by anticancer agent MNU, the degeneration of the photoreceptor cell layer always occurred starting from the central portion of the retina. Therefore, it could be found that, unlike a control group, the central portion of the retina showed a decrease in the cells of the photoreceptor cell layer and was changed into an irregular shape (FIG. 3A).

At the middle portion of the retina, the cell layer was better maintained than in a photograph of the central portion. However, it can be seen that there was a little damage to the cells (FIG. 3B).

It can be seen that the peripheral portion of the retina almost completely maintained its appearance (FIG. 3C).

The PTD-HspA1A was administered locally under the conjunctiva, and at 7 days after the local administration, observation was performed. The local administration was carried out for only 3 days from the first administration.

The central portion of the retina showed serious damage to the photoreceptor cell layer, but was conserved at a portion thereof. This clearly suggests that the PTD-HspA1A had an effect, as compared to the control group (FIG. 3D).

Referring to a photograph of the middle portion of the retina, it can be seen that the photoreceptor cells were better conserved as it goes toward the peripheral portion of the photograph, the normal photoreceptor cells could be more clearly observed than in the photograph of the central portion of the retina (FIG. 3E).

At the peripheral portion of the retina, the photoreceptor cell layer was conserved to an extent almost equal to the case of the systemic administration. Also, it can be seen that the peripheral portion was morphologically virtually normal (FIG. 3F).

The results show that PTD-HspA1A suppresses apoptosis in a retinal degeneration model.

### Example 6

### Organ Protection Effect in Organ Preservation Solution with PTD-HspA1A

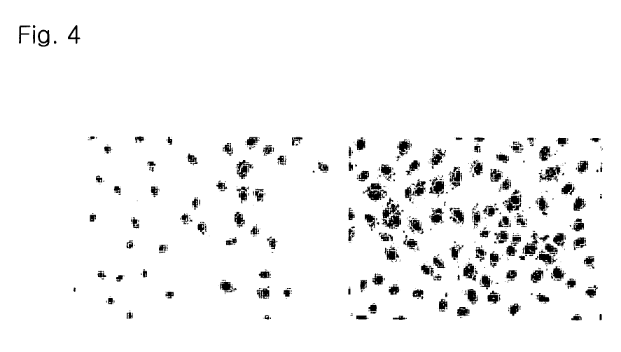

Isolated intestinal epithelial cells were divided into two groups, only one of which was given heat shock at 43° C. to induce the expression of the HspA1A protein. The cells were incubated at 37° C. for 2 hours, recovered and then stored in Wisconsin University solution at 4° C. for 24 hours. Then, the cells were incubated at 37° C. for 2 hours. The cells were fixed with 10% formalin, stained with hematoxilin & eosin and observed. It was observed that the cells of the group having the HspA1A protein expressed therein (FIG. 4, left photograph) were normally maintained, whereas the cells of the group having no HspA1A protein expressed therein showed the condensation of the nucleus and cytoplasm (FIG. 4, right photograph). The results show that HspA1A exhibits an organ-protecting effect in organ preservation solution.

### Example 7

### Apoptosis-Suppressing Effect of PTD-HspA1A on MSC Transplantation in Infarcted Myocardium

It is known that mesenchymal stem cell (MSC) therapy for myocardial injury has inherent limitations due to their poor viability after cell transplantation. It is reported that the survival rate of transplanted cells in an uninjured mouse heart is less than 1% at 4 days post transplantation (Toma, C., et al., Circulation 105:93-98 (2002)). Accordingly, there is a need to improve the survival of transplanted stem cells in an infarcted heart.

Generation of MSC. Bone marrow-derived mesenchymal stem cells (MCS) were harvested from femurs and tibia of 4-week old Sprague-Dawley male rats (about 100 g) by aspiration with 10 ml of MSC medium consisting of Dulbecco's modified Eagle's medium-low glucose supplemented with 10% fetal bovine serum and 1% antibiotic-penicillin and streptomycin solution. Bone marrow was isolated with Percoll-separation, and mononuclear cells were recovered. The recovered cells were washed twice and resuspended in 10% FBS-DMEM, and plated at 1×10⁶ cells/100 cm² in flasks. The cultures were maintained at 37° C. in a humidified atmosphere containing 5% CO2. After 48 or 72 hrs, the nonadherent cells were discarded, and the adherent cells were washed twice with PBS. The cultures were refreshed with fresh complete medium every 3 or 4 days for about 10 days. For further purification, the MSC were subjected to Isolex magnetic cell selection system (Nexell Therapeutics Inc. CA, USA). Briefly, the cell suspension was incubated with anti-CD34 monoclonal antibody, washed several times to have the unbound antibodies removed, and mixed with Dynabeads® M-450 coated with sheep anti-Mouse IgG, which recognizes the murine-derived anti-CD34 antibody. A magnetic field was applied to the chamber, enabling the CD34+ cell-bead complexes to be separated magnetically from the rest of the cell suspension. The remaining CD34-negative fraction was then further propagated. The cells were harvested with 0.25% trypsin and 1 mM EDTA for 5 min at 37° C., and replated on 100 cm² plates. On day 10 following the replating, the cells were quantified by the nonradioactive colorimetric assay WST-1 (Boehringer Mannheim) for an estimation of the proliferation rate. The quantification was based on the cleavage of tetrazolium salt, as recommended by the manufacturer, and showed that the process yielded 3×10⁶ cells with 95% purity.

Surgical procedure. Myocardial infarction was produced in male Sprague-Dawley rats (200±30 g) by surgical occlusion of the left anterior descending coronary artery (n=8 per group). The surgical process was performed under confocal microscopy. Briefly, rats were sedated and anesthetized for the procedure with ketamine (10 mg/kg) and xylazine (5 mg/kg), and the third and fourth ribs of the rats were cut to have their hearts exposed through the intercostal space. The left coronary artery was then ligated 2-3 mm from its origin with a 5-0 prolene suture (ETHICON, UK). After 60 minutes of occlusion, the hemostat was removed and snare released for reperfusion, with the ligature left loose on the surface of the heart. The wound was closed with a pulse-string suture. Throughout the operation, animals were ventilated with 95% O2 and 5% CO2 using a Harvard ventilator. Sham-operated animals were treated similarly, except that the coronary suture was not tied. Operative mortality in 48 hrs was 10%.

MSC transplantation. On day 48 after the induction of the infarction, animals that have survived the infarction were subjected to MSC transplantation. On the day of the transplantation, viable MSC were labeled with DAPI. Sterile DAPI solution was added into the culture medium at the final concentration of 50 μg/ml. The dye was allowed to remain in the culture dishes for 30 min, and the cells were rinsed 6 times with PBS to have the excess, unbound DAPI removed. Labeled cells were then detached with 0.25% (w/v) trypsin and suspended in serum-free medium for grafting. For the cell transplantation, MSC (2.0×105 cells) were suspended in 10 μl serum-free medium and injected into the region of infarction using a Hamilton syringe with a 30-gauge needle.

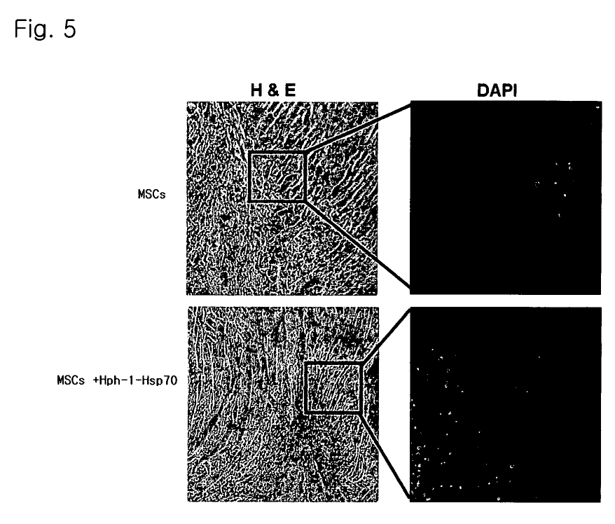

Detection of the implanted MSC. Four days following the implantation of the MSC, the animals were euthanized and fixed by transcardiac perfusion with 10% neutral buffered formaldehyde. The hearts were then isolated and immersed in 10% neural buffered formaldehyde for 24 hours. Each heart was then embedded in a paraffin block and sectioned to a thickness of 6 μm. The sections were then subjected to H&E and/or various immunohistochemical staining. As can be seen in FIG. 5, the H&E and the DAPI double staining shows that the Hph-1-HspA1A-treated heart is more populated with viable stem cells compared to the untreated one, indicating that the viable, mature cardiac myocytes have infiltrated into the scar area by 4 weeks after the implantation. The H&E stained sections show the border zone of the implanted cells and the host cardiomyocytes.

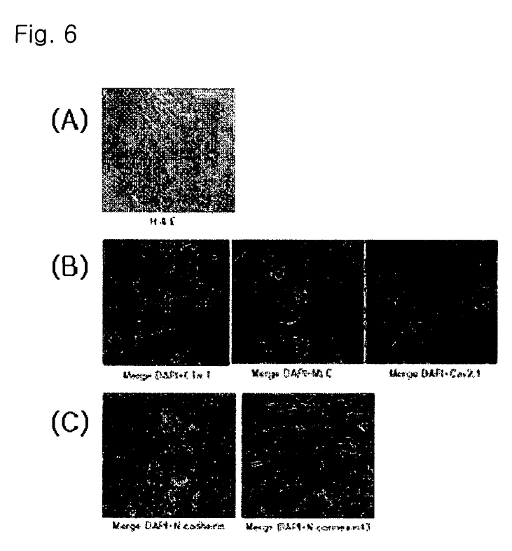

To confirm that the implanted cells were differentiated into cardiac myocyte-like cells, we showed by immunohistochemistry that the cardiac specific markers, CTn T, MHC, and Cav2.1 were detectible in the DAPI stained regions. DAPI-stained HspA1A-MSC in the host cardiomyocyte region also showed expression of connexin-43 and N-cadherin (FIG. 6).

Cardiac dimensions and performance parameters measured by transthoracic echocardiography are given in Table 1. At baseline (i.e., after infarction and before cell transplantation) echocardiographic parameters were not significantly different between the groups. The left ventricular end diastolic diameter (LVEDD), left ventricular end systolic diameter (LVESD), left ventricular end diastolic volume (LVEDV), and the left ventricular end systolic volume (LVESV) were significantly decreased in the HSPAIA-MSC group vs. the control group. The % fractional shortening (FS) and the % ejection fraction (EF) were significantly improved in the HSPA1A-MSC group compared to the control group. The transplantation of HSPA1A-MSC resulted in a further increase in the systolic performance (37.7% increment in % FS and 28.7% increment in % EF) compared with the MSC group. The peak circumferential and radial strain on infarct zone were increased in HspA1A-MSC group compared with any other group. The global circumferential and radial strain were also significantly increased in HspA1A-MSC group compared with any other group. These data suggested that transduction of PTD-HspA1A has a significant effect on the inhibitory mechanism for apoptosis.

TABLE 1
Echo-data in the Untreated control, MSC-treated, and the
HSPA1A-MSC-treated rats.

| | | | HspA1A- |
|---|---|---|---|
| Variables | Control (n = 8) | MSCs (n = 8) | MSCs (n = 8) |
| LVEDD, mm | 7.21 ± 0.50 | 6.72 ± 0.52 | 5.62 ± 0.40 |
| LVESD, mm | 6.11 ± 0.38 | 5.35 ± 0.50 | 3.71 ± 0.36 |
| FS, % | 14.63 ± 2.22 | 20.33 ± 1.95 | 27.99 ± 3.04 |
| LVEDV, ml | 0.84 ± 0.07 | 0.69 ± 0.19 | 0.53 ± 0.08 |
| LVESV, ml | 0.54 ± 0.08 | 0.37 ± 0.12 | 0.21 ± 0.07 |
| LVEF, % | 35.5 ± 4.8 | 47.0 ± 3.7 | 60.5 ± 4.1 |
| Peak S cir, % | −1.90 ± 0.40 | −4.31 ± 1.72 | −6.60 ± 1.25 |
| (infarct zone) | | | |
| Peak S rad, % | 3.91 ± 1.07 | 16.33 ± 1.40 | 20.04 ± 1.84 |
| (infarct zone) | | | |
| Global S cir, % | −4.23 ± 1.63 | −7.64 ± 1.27 | −12.05 ± 1.40 |
| Global S rad, % | 5.58 ± 2.42 | 25.14 ± 3.65 | 31.81 ± 3.75 |

Values are given as mean ± S.D.
LVEDD = left ventricular end diastolic diameter,
LVESD = left ventricular end systolic diameter,
FS = fractional shortening,
LVEDV = left ventricular end diastolic volume,
LVESV = left ventricular end systolic volume,
S cir = circumferential strain, and
S rad = radial strain.

### Example 8

### Apoptosis-Suppressing Effect of PTD-Hsc70

HSC70 (also referred to as HSP8A) is a constitutive member of the highly conserved heat shock protein 70 family, which generally comprises ˜1% of total cellular protein with possibly higher levels in transformed cells (Bakkenist, C. J., et al., Cancer Res. 59: 4219-4221 (1999)).

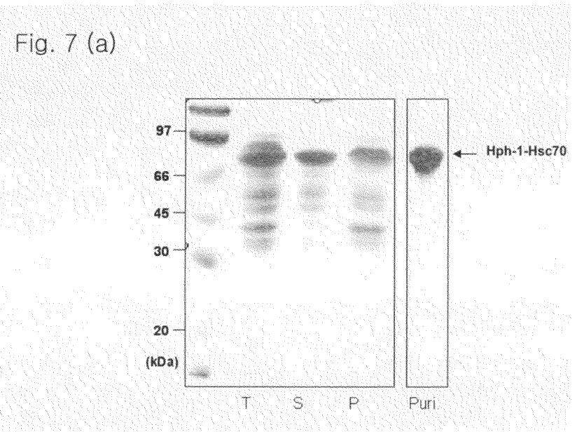

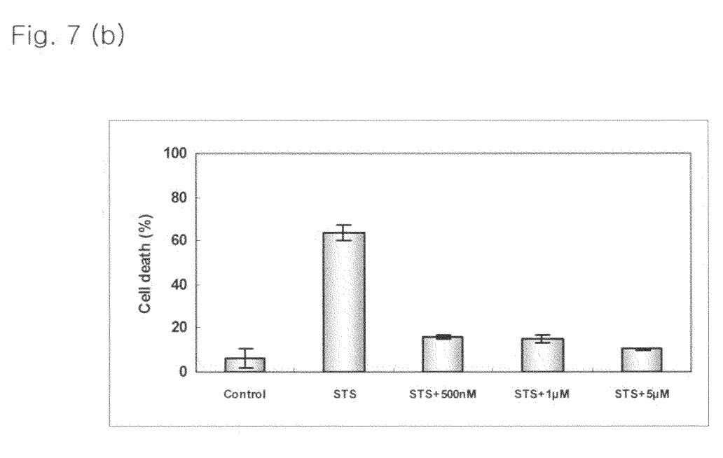

PTD-conjugated Hsc70 protein was purified as shown in FIG. 7A. Apoptosis was induced in Jurkat T cells by treating them with 0.5 μM staurosporin (STS). Subsequently, various concentrations of PTD-Hsc70 were added, and the cells were analyzed for the degree of apoptosis. The results show that the PTD-Hsc70 exhibits an apoptosis-suppressing effect in a concentration-dependent manner (FIG. 7B).

### Example 9

### Apoptosis-Suppressing Effect of PTD-cvHsp

cvHsp is a cardiovascular heat shock protein (Krief et al., J. Bio.l Chem. 274(51):36592-36600 (1999)).

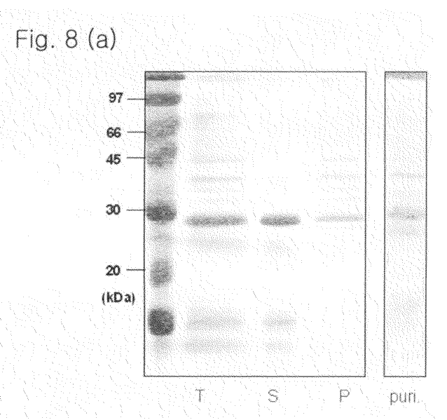

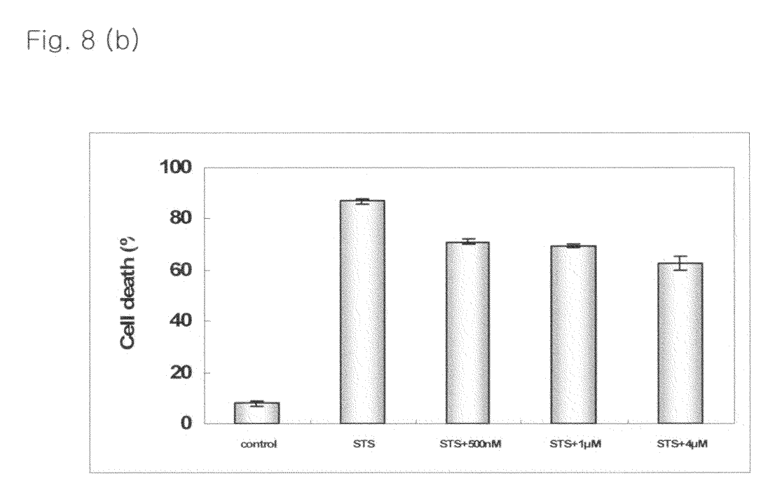

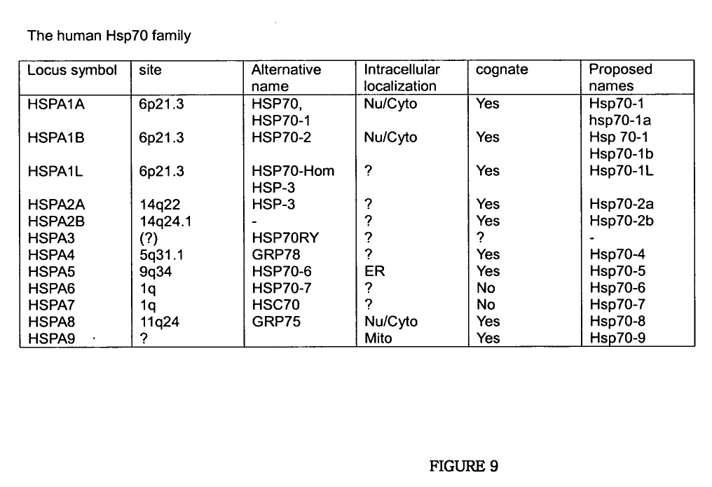

PTD-conjugated cvHsp protein was purified as shown in FIG. 8A. Apoptosis was induced in Jurkat T cells by treating them with 0.5 μM staurosporin (STS). Subsequently, various concentrations of PTD-Hsc70 were added, and the cells were analyzed for the degree of apoptosis. The results show that the PTD-cvHsp exhibits an apoptosis-suppressing effect in a concentration-dependent manner (FIG. 8B).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

What is claimed is:

1. A fusion protein comprising a protein transduction domain (PTD) and a heat shock protein (Hsp), wherein the Hsp is Hsp70 or cvHsp.

2. The fusion protein of claim 1, wherein said Hsp70 is selected from the group consisting of: HSPA1A, HSPA1B, HSPA1L, HSPA2A, HSPA2B, HSPA4, HSPA5, HSPA6, HSPA7, HSP8A and HSP9A.

3. The fusion protein of claim 1, wherein said Hsp70 is HSPA1A.

4. The fusion protein of claim 1, wherein said fusion protein comprises an Hsp70 amino acid sequence selected from the group consisting of:
(i) SEQ ID NO:11;
(ii) SEQ ID NO:12;
(iii) SEQ ID NO:13;
(iv) SEQ ID NO:14;
(v) SEQ ID NO:15;
(vi) SEQ ID NO:16;
(vii) SEQ ID NO:17;
(viii) SEQ ID NO:18;
(ix) SEQ ID NO:19;
(x) SEQ ID NO:20; and
(xi) SEQ ID NO:21.

5. The fusion protein of claim 1, wherein said fusion protein comprises the cvHsp amino acid sequence of SEQ ID NO:22.

6. The fusion protein of claim 1, wherein said fusion protein comprises the HspA1A amino acid sequence of SEQ ID NO:11.

7. The fusion protein of claim 1, wherein said PTD comprises an amino acid sequence selected from the group consisting of:
(i) SEQ ID NO:1;
(ii) SEQ ID NO:2;
(iii) SEQ ID NO:3;
(iv) SEQ ID NO:4;
(v) SEQ ID NO:5;
(vi) SEQ ID NO:6;
(vii) SEQ ID NO:7;
(viii) SEQ ID NO:8; and
(ix) SEQ ID NO:9.

8. The fusion protein of claim 1, wherein said PTD and said heat-shock protein are linked to each other by a direct covalent bond, a peptide bond, or a linker.

9. The fusion protein of claim 8, wherein said linker is a non-cleavage linker comprising 1 to 5 amino acids.

10. The fusion protein of claim 8, wherein said linker comprises Gly-Gly-Gly.

11. The fusion protein of claim 8, wherein said linker is a cleavage linker.

12. A pharmaceutical composition comprising the fusion protein of claim 1 in admixture with one or more pharmaceutically acceptable excipients.

13. The pharmaceutical composition of claim 12, further comprising at least one anti-platelet drug, anti-coagulant drug, anti-thrombotic drug, or an Hsp co-chaperone.

14. A fusion protein comprising a PTD and an amino acid sequence at least 95% identical to an amino acid sequence selected from the group consisting of:
(i) SEQ D NO:11;
(ii) SEQ ID NO:12;
(iii) SEQ ID NO:13;
(iv) SEQ ID NO:14;
(v) SEQ ID NO:15;
(vi) SEQ ID NO:16;
(vii) SEQ ID NO:17;
(viii) SEQ ID NO:18;
(ix) SEQ ID NO:19;
(x) SEQ ID NO:20;
(xi) SEQ ID NO:21; and
(xii) SEQ ID NO:22.

15. A method for prolonging cell, tissue or organ viability comprising contacting a cell population, tissue or organ with an amount of PTD-Hsp effective to suppress apoptosis in one or more cells of said cell population, tissue or organ, thereby prolonging the viability of said cell population, tissue or organ as compared to an untreated cell population, tissue or organ.

16. The method of claim 15, wherein said cell population, tissue or organ is contacted with the PTD-Hsp solution ex vivo or in vivo.

17. The method of claim 15, wherein the cells in said cell population are differentiated or precursor cells.

18. The method of claim 15, wherein the cells in said cell population are stem cells.

19. The method of claim 18, wherein said stem cells are hematopoietic stem cells, mesenchymal stem cells, stromal stem cells or neural stem cells.

20. The method of claim 19, wherein said hematopoietic stem cells are transplanted into an individual in need thereof, and wherein said hematopoietic stem cells are capable of differentiating into blood cells.

21. The method of 20, wherein said individual is a leukemia or blood cancer patient.

22. The method of 19, wherein said mesenchymal stem cells are transplanted into an individual in need thereof, and wherein said mesenchymal stem cells are capable of differentiating into osteocytes, chondrocytes, adipocytes or cardiomyocytes.

23. The method of claim 19, wherein said mesenchymal stem cells are transplanted into a heart.

24. The method of claim 23, wherein said heart is an infarcted heart.

25. The method of claim 23 or 24, wherein said mesenchymal stem cells are capable of differentiating into cardiomyocytes.

26. The method of claim 19, wherein said neural stem cells are transplanted into an individual in need thereof, and wherein said neural stem cells are capable of differentiating into nerve cells or non-nerve cells.

27. The method of claim 26, wherein said non-nerve cells are astrocytes or oligodendrocytes.

28. The method of claim 15, wherein the cells in said cell population are selected from the group consisting of: neural cells, fibroblasts, smooth muscle cells, tumor cells, haematopoietic cells, monocytes, macrophages, epithelial cells, keratinocytes, nerve cells, endothelial cells, granulocytes, erythrocytes, lymphocytes and platelets.

29. The method of claim 28, wherein said neural cells are neurons.

30. The method of claim 15, wherein the cells in said cell population are damaged cells and said contacting results in regeneration of said damaged cells.

31. The method of claim 15, wherein the cells in said cell population produce a product of interest, thereby increasing in vitro bioproduction of said product of interest.

32. The method of claim 15, wherein said contacting occurs during transfusions.

33. The method of claim 15, wherein said contacting occurs during transplantation of said cell population, tissue or organ.

34. The method of claim 30, wherein damage in said damaged cells, caused by reperfusion of said organ or tissue, is decreased.

35. The method of claim 15, wherein said contacting is by administering to a donor of said cell population, tissue or organ PTD-Hsp prior to or concurrent with removal of said cell population, tissue or organ.

36. The method of claim 35, wherein said organ is a solid organ.

37. The method of claim 36, wherein said solid organ is selected from heart, pancreas, kidney, lung or liver.

38. The method of claim 37, wherein said organ is a heart.

39. The method of claim 15, wherein said PTD-Hsp is in a solution.

40. The method of claim 39, wherein said solution is a hypothermic storage solution.

41. The method of claim 40, wherein said solution further comprises a concentration of a vitrification composition, wherein the vitrification occurs both within the cell population, tissue or organ, and in the solution.

42. A method of treating a pathological condition characterized by an elevated level of apoptosis, comprising administering to an individual in need of such treatment an amount of PTD-Hsp effective for treating the condition.

43. The method of claim 42, wherein the pathological condition is a stress-induced pathology.

44. The method of claim 43, wherein said stress-induced pathology is the result of an ischemic event.

45. The method of claim 44, wherein said ischemic event is selected from the group consisting of: a stroke due to ischemic cerebral infarction, ischemic acute renal failure, intestinal ischemia, ischemic heart disease due to myocardial infarction, myocardial ischemia and disorder after reperfusion, liver ischemia, brain ischemia, and ischemia retinae.

46. The method of claim 42, wherein said pathological condition is a chronic degenerative disease.

47. The method of claim 46, wherein said chronic degenerative disease is a neurodegenerative disease selected from the group consisting of: Alzheimer's disease, Parkinson's disease, Huntington's disease, multiple sclerosis, amyotrophic lateral sclerosis (ALS), spinobulbar atrophy, denervation atrophy, spinal muscular dystrophy (SMA), pigmentary degeneration of the retina and glaucoma, cerebellar degeneration and neonatal jaundice, otosclerosis, stroke, dementia, and successive delayed neuronal death (DND).

48. The method of claim 46, wherein said chronic degenerative disease is degenerative atrophy.


SEQUENCE LISTING (22 sequences)

SEQ ID NO: 1 - protein, 11 aa, Homo sapiens
YARVRRRGPRR

SEQ ID NO: 2 - protein, 9 aa, Homo sapiens
AKAARQAAR

SEQ ID NO: 3 - protein, 11 aa, HIV
YGRKKRRQRRR

SEQ ID NO: 4 - protein, 16 aa, Drosophila sp.
RQIKIWFQNRRMKWKK

SEQ ID NO: 5 - protein, 34 aa, HSV
DAATATRGRSAASRPTERPRAPARSASRPRRPVE

SEQ ID NO: 6 - protein, 7 aa, Homo sapiens
RRRRRRR

SEQ ID NO: 7 - protein, 26 aa, Homo sapiens
AAVALLPAVLLALLAPAAADQNQLMP

SEQ ID NO: 8 - protein, 21 aa, Homo sapiens
KETWWETWWTEWSQPKKKRKV

SEQ ID NO: 9 - protein, 21 aa, Homo sapiens
KETWFETWFTEWSQPKKKRKV

SEQ ID NO: 10 - DNA, 1926 bp, Homo sapiens
atggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggacaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattag

SEQ ID NO: 11 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGDTKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 12 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKASKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 13 - protein, 641 aa, Homo sapiens
MATAKGIAIGIDLGTTYSCVGVFQHGKVEIIANDQGNRTTPSYVAFTDTERLIGDAAKNQVAMNPQNTVFDAKRLIGRKFNDPVVQADMKLWPFQVINEGGKPKVLVSYKGENKAFYPEEISSMVLTKLKETAEAFLGHPVTNAVITVPAYFNDSQRQATKDAGVIAGLNVLRIINEPTAAAIAYGLDKGGQGERHVLIFDLGGGTFDVSILTIDDGIFEVKATAGDTHLGGEDFDNRLVSHFVEEFKRKHKKDISQNKRAVRRLRTACERAKRTLSSSTQANLEIDSLYEGIDFYTSITRARFEELCADLFRGTLEPVEKALRDAKMDKAKIHDIVLVGGSTRIPKVQRLLQDYFNGRDLNKSINPDEAVAYGAAVQAAILMGDKSEKVQDLLLLDVAPLSLGLETAGGVMTALIKRNSTIPTKQTQIFTTYSDNQPGVLIQVYEGERAMTKDNNLLGRFDLTGIPPAPRGVPQIEVTFDIDANGILNVTAMDKSTGKVNKITITNDKGRLSKEEIERMVLDAEKYKAEDEVQREKIAAKNALESYAFNMKSVVSDEGLKGKISESDKNKILDKCNELLSWLEVNQLAEKDEFDHKRKELEQMCNPIITKLYQGGCTGPACGTGYVPGRPATGPTIEEVD

SEQ ID NO: 14 - protein, 639 aa, Homo sapiens
MSARGPAIGIDLGTTYSCVGVFQHGKVEIIANDQGNRTTPSYVAFTDTERLIGDAAKNQVAMNPTNTIFDAKRLIGRKFEDATVQSDMKHWPFRVVSEGGKPKVQVEYKGETKTFFPEEISSMVLTKMKEIAEAYLGGKVHSAVITVPAYFNDSQRQATKDAGTITGLNVLRIINEPTAAAIAYGLDKKGCAGGEKNVLIFDLGGGTFDVSILTIEDGIFEVKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNALESYTYNIKQTVEDEKLRGKISEQDKNKILDKCQEVINWLDRNQMAEKDEYEHKQKELERVCNPIISKLYQGGPGGGSGGGGSGASGGPTIEEVD

SEQ ID NO: 15 - protein, 639 aa, Homo sapiens; identical to SEQ ID NO: 14

SEQ ID NO: 16 - protein, 840 aa, Homo sapiens
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVREYVYEMRDKLSGEYEKFVSEDGRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 17 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 18 - protein, 643 aa, Homo sapiens
MQAPRELAVGIDLGTTYSCVGVFQQGRVEILANDQGNRTTPSYVAFTDTERLVGDAAKSQAALNPHNTVFDAKRLIGRKFADTTVQSDMKHWPFRVVSEGGKPKVRVCYRGEDKTFYPEEISSMVLSKMKETAEAYLGQPVKHAVITVPAYFNDSQRQATKDAGAIAGLNVLRIINEPTAAAIAYGLDRRGAGERNVLIFDLGGGTFDVSVLSIDAGVFEVKATAGDTHLGGEDFDNRLVNHFMEEFRRKHGKDLSGNKRALRRLRTACERAKRTLSSSTQATLEIDSLFEGVDFYTSITRARFEELCSDLFRSTLEPVEKALRDAKLDKAQIHDVVLVGGSTRIPKVQKLLQDFFNGKELNKSINPDEAVAYGAAVQAAVLMGDKCEKVQDLLLLDVAPLSLGLETAGGVMTTLIQRNATIPTKQTQTFTTYSDNQPGVFIQVYEGERAMTKDNNLLGRFELSGIPPAPRGVPQIEVTFDIDANGILSVTATDRSTGKANKITITNDKGRLSKEEVERMVHEAEQYKAEDEAQRDRVAAKNSLEAHVFHVKGSLQEESLRDKIPEEDRRKMQDKCREVLAWLEHNQLAEKEEYEHQKRELEQICRPIFSRLYGGPGVPGGSSCGTQARQGDPSTGPIIEEVD

SEQ ID NO: 19 - protein, 247 aa, Homo sapiens
MQAPRELAVGIDLGTTYSCVGVFQQGRVEILANDQGNRTTPSYVAFTDTERLVGDAAKNQAALNPHNTVFDAKRLIGRKFADTTVQSDMKHWPFKVVSGGGKPKVRVCYRGEDKTFYPEEISSMVLTKMKETAEAYLGQPVKHAVITVPTYFSNSQRQATKDAGAIAGLKVLPIINEATAAAIAYGLDRRRAGKRNVLIFDLGGGTFDVSVLTIDAGVFEVKATAGDTHLGGEDFDNRLVNHFMEEF

SEQ ID NO: 20 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 21 - protein, 674 aa, Homo sapiens
MISASRAAARLPLLLPRGGPVPAVPGLAQTFWNGLSQNVLRAASSRKYASEAIKGAVIGIDLGTTNSCVAVMEGKQAKVLENSEGARTTPSVVAFTADGERLVGMPAKRQAVTNPHNTFYATKRLIGRRFDDSEVKKDIKNVPFKIVRASNGDAWVEAHGKLYSPSQIGAFVLMKMKETAENYLGHPAKNAVITVPAYFNDSQRQATKDAGQISGLNVLRVINEPTAAALAYGLDKSEDKIIAVYDLGGGTFDISILEIQKGVFEVKSTNGDTFLGGEDFDQALLQYIVKEFKRETSVDLTKDNMALQRVREASEKAKCELSSSVQTDINLPYLTMDASGPKHLNMKLSRSQFEGIVADLIKRTVAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMASDNKLLGQFTLVGIPPAPRGVPQIEVTFDIDANGIVHVSAKDKGTGREQQIVIQSSGGLSKDEIENMVKNAEKYAEEDRRRKERVEAVNLAEGIIHDTESKMEEFKDQLPADECNKLKEEIAKMRELLARKDTETGENIRQAATSLQQASLKLFEMAYKKMASERESSGSSGDQKEEK

SEQ ID NO: 22 - protein, 170 aa, Homo sapiens
MSHRTSSTFRAERSFHSSSSSSSSSTSSSASRALPAQDPPMEKALSMFSDDFGSFMRPHSEPLAFPARPGGAGNIKTLGDAYEFAVDVRDFSPEDIIVTTSNNHIEVRAEKLAADGTVMNTFAHKCQLPEDVDPTSVTSALREDGSLTIRARRHPHTEHVQQTFRTEIKI